(19) United States
(12) Patent Application Publication
(10) Pub. No.: US 2008/0085885 A1
(43) Pub. Date: Apr. 10, 2008

(54) Inhibition Of Voluntary Ethanol Consumption With Non-Peptidyl Melanocortin-4 Receptor Agonists

(76) Inventors: Linda Bristow, Boston, MA (US); Tung M. Fong, Somerset, NJ (US); Andrew C. Morse, San Diego, CA (US); Kunkun Ren, San Diego, CA (US)

Correspondence Address: MERCK AND CO., INC, P O BOX 2000, RAHWAY, NJ, 07065-0907, US

(21) Appl. No.: 11/660,117

(22) PCT Filed: Aug. 9, 2005
(86) PCT No.: PCT/US05/28128

§ 371 Date: Feb. 12, 2007

Related U.S. Application Data
(60) Provisional application No. 60/601,486, filed on Aug. 13, 2004.

(51) Int. Cl. A61K 31/454; A61K 31/506; A61P 25/32

(52) U.S. Cl. 514/210.18; 514/256; 514/326

(57) ABSTRACT

The present invention relates to methods of inhibiting or reducing voluntary alcohol consumption in a subject comprising administering a non-peptidyl melanocortin 4 receptor agonist to said subject. The present invention further relates to methods of treating or preventing alcoholism, alcohol abuse, and alcohol related disorders in a subject comprising administering a non-peptidyl melanocortin 4 receptor agonist to said subject. The present invention further provides for pharmaceutical compositions and medicaments useful in carrying out these methods.

### BACKGROUND OF THE INVENTION

Alcohol abuse is one of the most significant problems in modern society. Nearly 14 million people in the United States, approximately 1 in every 13 adults, abuse alcohol or are alcoholics (U.S. Dept. of Human Services, 2001 National Household Survey on Drug Abuse: Volume 1 (BKD461, SMA 02-3758)). According to the National Institutes of Health, each year alcohol abuse accounts for 45% of all car crash fatalities (over 20,000 individuals) and is involved in approximately 44% of all short-stay hospital visits. An additional 26,000 individuals die from alcohol-associated chronic liver disease and cirrhosis of the liver (NCHS, National Vital Statistics Report Vol. 50, No. 5, 2000). The Justice Department reported that alcohol was involved in nearly 40% of all violent crimes in 1998. The resulting economic cost of alcohol abuse to the United States is estimated to be nearly $150 billion per year.

The causes of alcoholism are not fully known. Genetics may play a role; a family history of alcoholism makes it more likely for a person to develop alcoholism if that person chooses to drink. Certain environmental risk factors may also influence whether a person with a genetic risk for alcoholism ever develops the disease.

Alcohol problems may be classified into two categories, alcoholism or alcohol dependence, and alcohol abuse. Alcoholism is a dependence on alcohol and is characterized by abnormal alcohol seeking behavior that leads to impaired control over drinking. Alcohol abuse is characterized by drinking too much or too often, without being an alcoholic. Alcohol misuse has also been found to predispose the subject to osteoporosis, slow bone healing, impaired wound healing, inhibited osteoblastic function and diminished immune defenses. Alcohol intoxication increases the risk of further accidents, and decreases the pain inhibition that would make a normal patient more careful. Alcohol dependence also leads to altered cognitive and emotional functions, such as impaired judgment, feelings of incompetency, low self-esteem, despair in relationships, feelings of failure, and depression.

Several medications are currently used to treat alcoholism. Disulfuram (Antabuse®) and Naltrexone (Trexan®) are the only FDA approved products that are currently available for adjunctive use in the treatment of alcohol abuse. Disulfuram works by blocking the intermediary metabolism of alcohol in the body to produce a build up of acetaldehyde, which in turn produces markedly adverse behavioral and physiological effects, such as severe nausea and vomiting. Patient compliance in taking the drug is poor due to these side effects (see T W Rall, in: Goodman and Gilman's The Pharmacological Basis of Therapeutics, A G Gilman et al, 8th Edition, Chap 17, pp 378-379). Naltrexone is a well-known opioid receptor antagonist and is thought to reduce the positive reinforcing effects of ethanol by blocking the effects of endogenous opioids and reducing alcohol stimulated dopamine release. In practice, naltrexone is only moderately effective because it is relatively short acting and patients require co-treatment with behavioral therapy for the drug to have any effect (J R Volpicelli et al, Arch Gen Psychiatry, 1992, 49:876-880). Acamprosate, which is not currently approved for use in the United States, is believed to minimize the negative side-effects associated with protracted abstinence by potentiating GABA and attenuating glutamate release. Benzodiazepines (Valium®, Librium®) are also sometimes useful during the first days after patients stop drinking to help them safely withdraw from alcohol; however, these medications can not be used for longer periods because benzodiazepines may also produce dependence in patients. As a result, there is a continuing need to develop new compounds that are useful for the treatment of alcoholism and alcohol abuse in mammals.

Recent studies support a role for melanocortin signaling in behavioral and neurochemical actions of ethanol. The melanocortin (MC) system is composed of peptides that are cleaved from the polypeptide precursor, proopiomelanocortin (POMC). These peptides include adrenocorticotropic hormone (ACTH), α-melanocyte stimulating hormone (α-MSH), β-MSH, and γ-MSH. Brain melanocortin peptides are produced primarily by neurons within the hypothalamic arcuate nucleus, the nucleus of the solitary tract, and the medulla. Genetic and pharmacological evidence reveals that melanocortin signaling is involved with grooming behavior, antipyretic and anti-inflammatory responses, learning, reproductive function, and regulation of appetite and energy homeostasis.

A recent report found significant differences in brain melanocortin 3 and melanocortin 4 receptor levels between rats selectively bred for high ethanol consumption (AA) and low ethanol consumption (ANA). AA rats selectively bred for high ethanol drinking have lower levels of melanocortin 3 receptor in the shell of the nucleus accumbens when compared with controls, but have high levels of melanocortin 3 receptor (MC3R) and melanocortin 4 receptor (MC4R) in various regions of the hypothalamus (Lindblom et al., Phann Biochein Behav 72:491-496, 2002). It has been shown that central infusion of the non-selective melanocortin agonist, MTII, significantly reduces voluntary ethanol drinking and prevents ethanol-induced changes in endogenous opioid peptide levels in the substantia nigra and VTA of AA rats with an established ethanol intake (Ploj et al., Brain Res Bull, 59:97-104, 2002). It was suggested that melanocortin signaling may regulate ethanol drinking by modulating endogenous opioid activity within mesolimbic dopamine pathways (Ploj et al., Brain Res Bull, 59:97-104, 2002). However, a recent report indicated that a MC4R-selective antagonist (HS014) has no effect on ethanol drinking by AA rats (Ploj et al., Brain Res Bull, 59:97-104, 2002).

It is unclear which melanocortin receptor(s) are important for modulating MTII-induced reductions of ethanol consumption (Ploj et al., Brain Res Bull, 59:97-104, 2002). MTII is a non-selective melanocortin agonist that binds, with varying affinity, to all centrally expressed melanocortin receptors (MC1R, MC2R, MC3R, MC4R and MC5R), but has the greatest affinity for MC3R and MC4R (Haskell-Luevano et al., J. Med. Chem. 40:1738-1748, 1997; Schioth et al., Peptides, 18:1009-1013, 1997). It is also possible that melanocortin 1 receptor (MC1R) and/or melanocortin 5 receptor (MC5R) are involved, as MTII binds to both of these receptors (Haskell-Luevano et al., J. Med. Chem. 40:1738-1748, 1997; Schioth et al., Peptides, 18:1009-1013, 1997). MC1R is expressed specifically in periaqueductal gray (PG) region of the brain (Xia et al., NeuroReport, 6:2193-2196, 1995) while MC5R is found in several brain regions, including the NAc (Griffon et al., Biochem Biophys Res Com, 200:1007-1014, 1994).

There is a need for an effective therapy for the treatment or prevention of alcoholism, alcohol abuse and alcohol related disorders.

It has now been found that non-peptidyl melanocortin 4 receptor agonists are useful to inhibit alcohol self-administration and consumption. The selective melanocortin 4 receptor agonists of the present invention are beneficial over non-selective melanocortin agonists since selective MC4R agonists do not exhibit the side effects associated with non-selective MC4R agonists, such as melanocortin 1 mediated pigment changes and worsening of acne associated with MC5R agonists. Additionally, the use of non-peptidyl melanocortin-4 receptor agonists for the treatment of alcoholism and related disorders is beneficial over the use of peptidyl melanocortin 4 agonists since peptidyl MC4R agonists are typically degraded in the stomach and GI tract, and are therefore not orally bioavailable, and brain penetration of small molecules is better.

It is an object of the present invention to identify methods of inhibiting alcohol consumption comprising administering a non-peptidyl melanocortin 4 receptor agonist to a subject. It is another object of the present invention to identify methods of treating alcoholism and alcohol abuse comprising administering a non-peptidyl melanocortin 4 receptor agonist to a subject. It is yet another object of the invention to identify methods of preventing alcoholism, alcohol abuse, and alcohol-related disorders. It is a further object of the present invention to provide a method of manufacture of a medicament useful to inhibit alcohol consumption.

### SUMMARY OF THE INVENTION

The present invention provides a method of inhibiting alcohol consumption comprising administration of a therapeutically effective amount of a non-peptidyl melanocortin 4 receptor agonist to a subject.

The present invention further provides a method of reducing alcohol consumption comprising administration of a therapeutically effective amount of a non-peptidyl melanocortin 4 receptor agonist to a subject.

The present invention further provides a method of preventing alcohol consumption comprising administration of a therapeutically effective amount of a non-peptidyl melanocortin 4 receptor agonist to a subject.

The present invention also provides a method of treating or preventing alcoholism comprising administration of a therapeutically effective amount of a non-peptidyl melanocortin 4 receptor agonist to a subject.

The present invention also provides a method of treating or preventing alcohol abuse comprising administration of a therapeutically effective amount of a non-peptidyl melanocortin 4 receptor agonist to a subject.

The present invention further provides a method of treating or preventing alcohol-related diseases comprising administration of a therapeutically effective amount of a non-peptidyl melanocortin 4 receptor agonist to a subject.

The present invention is also concerned with treatment of these conditions, and the use of the compounds and compositions of the present invention for manufacture of a medicament useful for treating these conditions.

### BRIEF DESCRIPTION OF THE DRAWINGS

FIG. 1. Operant training for oral ethanol across 30 minute test sessions in rats (n=8-10). Rats were trained on a continuous reinforcement (FR-1) schedule using a saccharine fading procedure and once stable levels of ethanol self-administration (10% w/v) were achieved, testing was initiated. Test 1 examined the effects of melanocortin 4 receptor agonist, Compound A, on maintenance levels of ethanol self-administration and Test 2 examined the effects of Compound A on the Alcohol Deprivation Effect (ADE).

FIG. 2. Animal Model of Alcohol Reward: shows the mean ethanol consumed in a 30 minute discrete trial session. Operant training for oral ethanol in 30 minute test sessions in rats (n=8-10). Melanocortin 4 receptor agonist, Compound A, was administered at 0, 5, 10 or 20 mg/kg, po prior to testing. A significant reduction in ethanol self-administration was observed following 20 mg/kg. Water consumption was not significantly changed following any dose of Compound A. Ethanol consumed is represented as means ±SEM. The symbol * indicates a significant difference between the vehicle and drug treated groups (p<0.05).

FIG. 3. Animal Model of Alcohol Relapse: shows the attenuation of the Alcohol Deprivation Effect (ADE). Operant training for oral ethanol in 30 minute test sessions in rats (n=8-10). Following the maintenance self-administration test, rats were rebaselined prior to being confined to their home-cages for a 10 day deprivation period. Melanocortin 4 receptor agonist, Compound A was administered at 0, 5, 10 or 20 mg/kg, po prior to reintroduction to the test chambers following deprivation. A significant attenuation of the alcohol deprivation effect was observed following 5 and 10 mg/kg and a further reduction below baseline levels was observed following 20 mg/kg. Water consumption was not significantly changed following any dose of Compound A. Ethanol consumed is represented as means ±SEM. The symbol * indicates a significant difference between baseline and drug treated groups (p<0.05). The symbol # indicates a significant difference between vehicle treated (ADE) and drug treated groups (p<0.05).

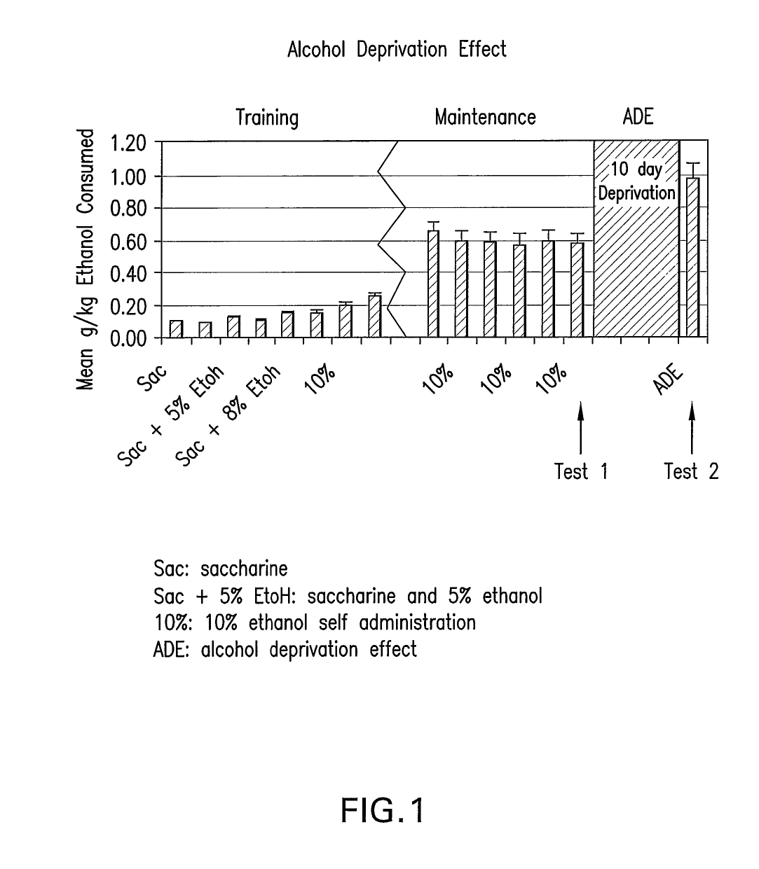

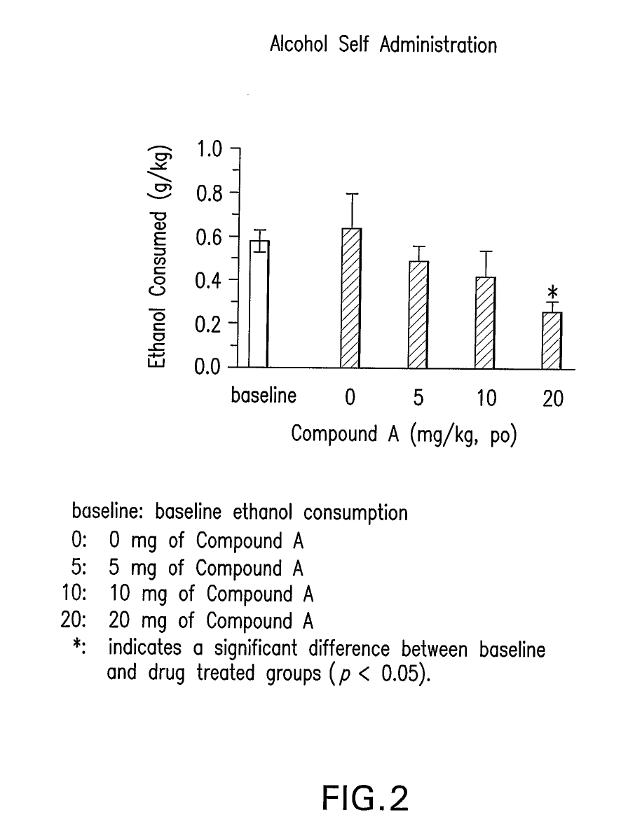

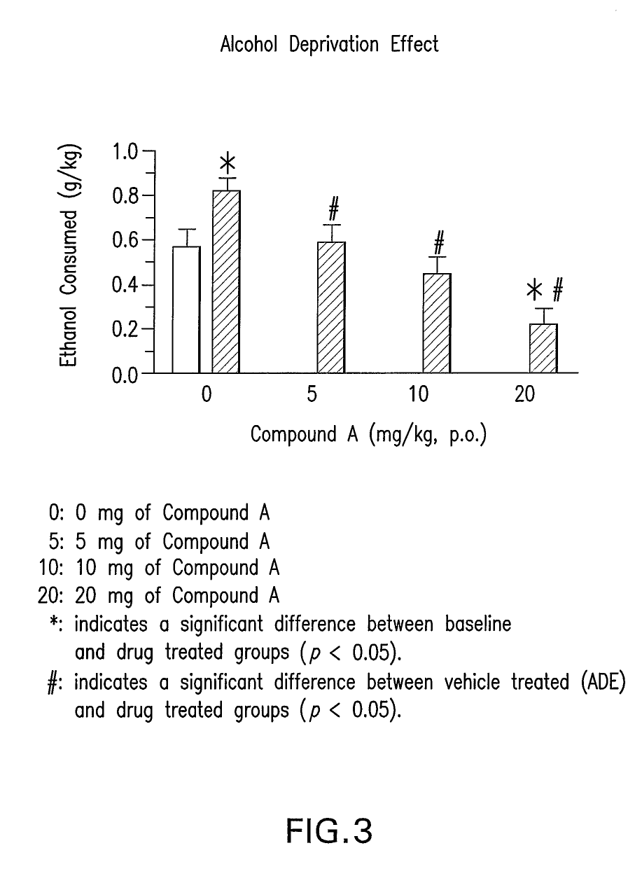

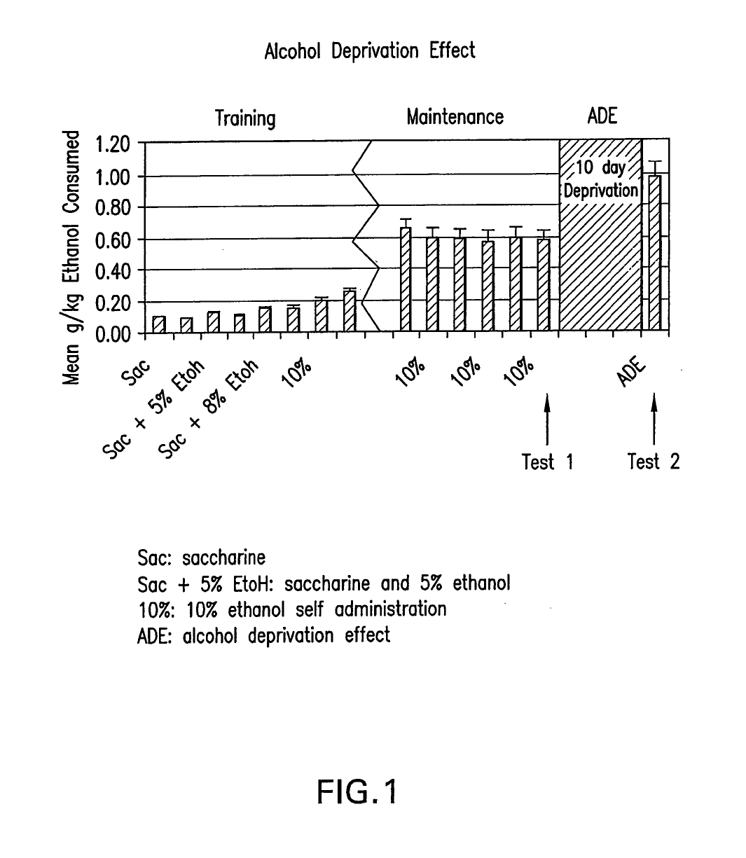

### DETAILED DESCRIPTION OF THE INVENTION

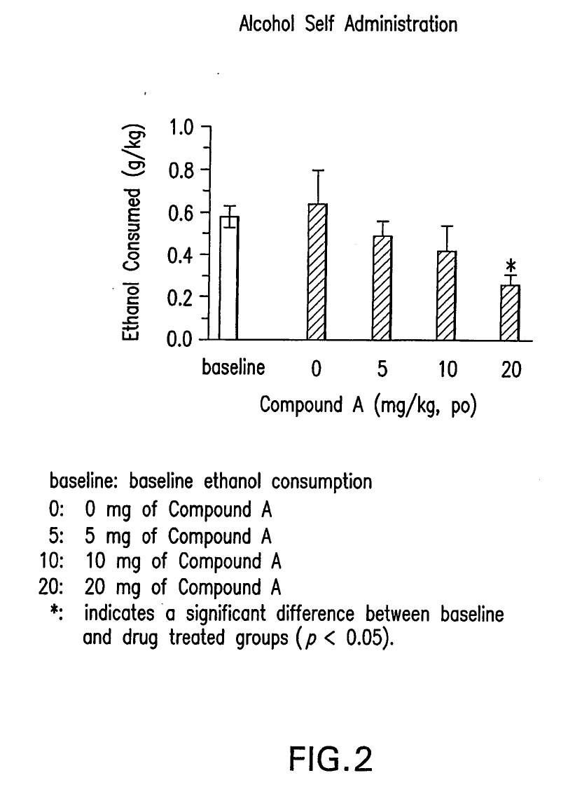

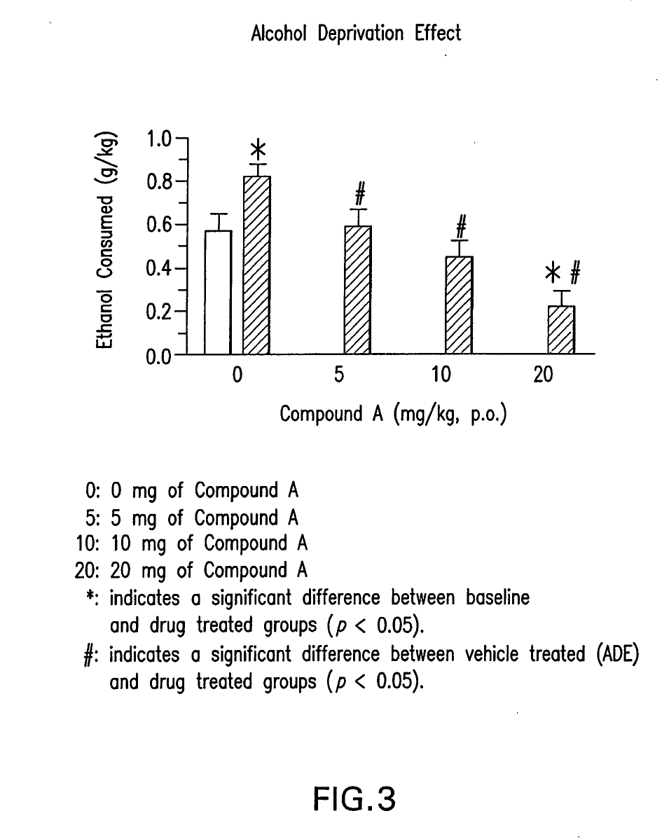

The present invention shows that nonpeptidyl, selective melanocortin 4 receptor agonists inhibit alcohol self-administration and consumption. It was found that the selective melanocortin-4 receptor agonist, Compound A, dose dependently reduced ethanol self administration by approximately 50% in male Wistar rats (See FIG. 2). It was also found that Compound A reduced the increase in ethanol consumption typically seen after a 10 day period of forced deprivation (the alcohol deprivation effect (ADE)) in male Wistar rats (See FIG. 3). Alcohol deprivation effect (ADE) is the transient increase in oral ethanol self administration following forced deprivation, and is characterized by loss of control over intake. ADE is precipitated by re-exposure to alcohol related cues after a period of alcohol deprivation. These studies show that selective melanocortin 4 receptor agonists block the acute reinforcing effects of alcohol. As a result, treatment with a selective melanocortin agonist resulted in a significant reduction of voluntary ethanol consumption in rats, as well as a prevention of the significant elevation in consumption observed after a brief deprivation period (ADE). Additionally, MC4R agonist Compound A had no significant effects on water consumption suggesting its effects on consumption are specific to ethanol and not a general decrease in consummatory behavior.

The present invention provides a method of inhibiting alcohol self administration. The present invention also provides a method of inhibiting alcohol consumption. The present invention also provides a method of inhibiting alcohol consumption in a subject currently consuming alcohol. The present invention further provides a method of reducing alcohol consumption. The present invention further provides a method of reducing alcohol consumption in a subject currently consuming alcohol. The present invention further provides a method of treating or preventing alcoholism. The present invention further provides a method of treating or preventing alcohol abuse. The present invention also provides a method of treating or preventing alcohol-related disorders. The present invention further provides a significant reduction in voluntary alcohol consumption. The present invention further provides a method of preventing a relapse of alcohol consumption at pre-treatment levels following a period of alcohol deprivation. The present invention further provides a method of reducing the amount of alcohol consumed as the result of a relapse of alcohol consumption. The present invention also relates to pharmaceutical compositions, and medicaments useful for carrying out these methods.

The methods of the present invention comprise a non-peptidyl melanocortin 4 receptor agonist. The melanocortin 4 receptor agonist of use in the present invention is selected from any non-peptidyl melanocortin 4 receptor agonist known in the art. Additionally, the non-peptidyl melanocortin 4 receptor agonist may be a selective melanocortin 4 agonist. For convenience, the use of an orally active non-peptidyl selective melanocortin 4 receptor agonist is preferred. To facilitate dosing, it is preferred that the melanocortin 4 receptor agonist is a long acting melanocortin 4 receptor agonist.

In one embodiment of the present invention, the invention is directed to a method of inhibiting alcohol consumption comprising administering a therapeutically effective amount of a non-peptidyl melanocortin 4 receptor agonist, or a pharmaceutically acceptable salt thereof, to a subject in need thereof. In another embodiment of the present invention, the invention is directed to a method of inhibiting alcohol consumption comprising administering a therapeutically effective amount of a non-peptidyl selective melanocortin 4 receptor agonist, or a pharmaceutically acceptable salt thereof, to a subject in need thereof. In another embodiment of the present invention, the invention is directed to a method of inhibiting the alcohol deprivation effect comprising administering to a subject in need thereof a therapeutically effective amount of a non-peptidyl selective melanocortin 4 receptor agonist, or a pharmaceutically acceptable salt thereof. In another embodiment of the present invention, the invention is directed to a method of inhibiting alcohol consumption in a subject currently consuming alcohol comprising administering to the subject a therapeutically effective amount of a non-peptidyl selective melanocortin 4 receptor agonist, or a pharmaceutically acceptable salt thereof.

In another embodiment of the present invention, the invention is directed to a method of inhibiting the alcohol deprivation effect in a subject currently consuming alcohol comprising administering to the subject a therapeutically effective amount of a non-peptidyl selective melanocortin 4 receptor agonist, or a pharmaceutically acceptable salt thereof.

In another embodiment of the present invention, the invention is directed to a method of reducing alcohol consumption comprising administering a therapeutically effective amount of a non-peptidyl melanocortin 4 receptor agonist, or a pharmaceutically acceptable salt thereof, to a subject in need thereof. In a class of this embodiment, the reduction of alcohol consumption is the reduction of voluntary alcohol consumption.

In another embodiment of the present invention, the invention is directed to a method of reducing the amount of alcohol consumed as the result of a relapse of alcohol consumption comprising administering a therapeutically effective amount of a non-peptidyl melanocortin 4 receptor agonist, or a pharmaceutically acceptable salt thereof, to a subject in need thereof.

In another embodiment of the present invention, the invention is directed to a method of reducing alcohol consumption in a subject currently consuming alcohol comprising administering to the subject a therapeutically effective amount of a non-peptidyl melanocortin 4 receptor agonist, or a pharmaceutically acceptable salt thereof. In another embodiment of the present invention, the invention is directed to a method of reducing alcohol consumption in a subject currently consuming alcohol comprising administering to the subject a therapeutically effective amount of a non-peptidyl selective melanocortin 4 receptor agonist, or a pharmaceutically acceptable salt thereof.

In another embodiment of the present invention, the invention is directed to a method of treating or preventing alcoholism comprising administering a therapeutically effective amount of a non-peptidyl melanocortin 4 receptor agonist, or a pharmaceutically acceptable salt thereof, to a subject in need thereof.

In another embodiment of the present invention, the invention is directed to a method of treating or preventing alcohol abuse comprising administering a therapeutically effective amount of a non-peptidyl melanocortin 4 receptor agonist, or a pharmaceutically acceptable salt thereof, to a subject in need thereof.

In another embodiment of the present invention, the invention is directed to a method of treating or preventing alcohol-related disorders comprising administering a therapeutically effective amount of a non-peptidyl melanocortin 4 receptor agonist, or a pharmaceutically acceptable salt thereof, to a subject in need thereof.

In another embodiment of the present invention, the invention is directed to a method of preventing a relapse of alcohol consumption to pre-treatment levels following a period of alcohol deprivation comprising administering a therapeutically effective amount of a non-peptidyl melanocortin 4 receptor agonist, or a pharmaceutically acceptable salt thereof, to a subject in need thereof.

In another embodiment of the present invention, the invention is directed to a method of maintaining abstinence in a subject in need thereof comprising administering to the subject a therapeutically effective amount of a non-peptidyl selective melanocortin 4 receptor agonist, or a pharmaceutically acceptable salt thereof.

In another embodiment of the present invention, the invention is directed to a method of reducing alcohol consumption comprising administering to a subject a therapeutically effective amount of a non-peptidyl selective melanocortin 4 receptor agonist, or a pharmaceutically acceptable salt thereof, wherein the functional activity of the melanocortin 4 receptor agonist is characterized by an EC₅₀ at least 14-fold more selective for the human melanocortin 4 receptor than for the human melanocortin 1 receptor, human melanocortin 2 receptor, the human melanocortin 3 receptor and the human melanocortin 5 receptor. In another embodiment of the present invention, the invention is directed to a method of reducing alcohol consumption comprising administering to a subject a therapeutically effective amount of a non-peptidyl selective melanocortin 4 receptor agonist, or a pharmaceutically acceptable salt thereof, wherein the functional activity of the melanocortin 4 receptor agonist is characterized by an EC₅₀ at least 65-fold more selective for the human melanocortin 4 receptor than for the human melanocortin 1 receptor, human melanocortin 2 receptor, the human melanocortin 3 receptor and the human melanocortin 5 receptor. In another embodiment of the present invention, the invention is directed to a method of reducing alcohol consumption comprising administering to a subject a therapeutically effective amount of a non-peptidyl selective melanocortin 4 receptor agonist, or a pharmaceutically acceptable salt thereof, wherein the functional activity of the melanocortin 4 receptor agonist is characterized by an EC₅₀ at least 200-fold more selective for the human melanocortin 4 receptor than for the human melanocortin 1 receptor, human melanocortin 2 receptor, the human melanocortin 3 receptor and the human melanocortin 5 receptor.

In a class of these embodiments, the functional activity of the melanocortin 4 agonist is characterized by an EC₅₀ at least 14-fold more selective for the human melanocortin 4 receptor than for the human melanocortin 1 receptor. In another class of these embodiments, the functional activity of the melanocortin 4 agonist is characterized by an EC₅₀ at least 120-fold more selective for the human melanocortin 4 receptor than for the human melanocortin 1 receptor.

In another class of these embodiments, the functional activity of the melanocortin 4 agonist is characterized by an EC₅₀ at least 100-fold more selective for the human melanocortin 4 receptor than for the human melanocortin 2 receptor. In another class of these embodiments, the functional activity of the melanocortin 4 agonist is characterized by an EC₅₀ at least 700-fold more selective for the human melanocortin 4 receptor than for the human melanocortin 2 receptor.

In another class of these embodiments, the functional activity of the melanocortin 4 agonist is characterized by an EC₅₀ at least 30-fold more selective for the human melanocortin 4 receptor than for the human melanocortin 3 receptor. In another class of these embodiments, the functional activity of the melanocortin 4 agonist is characterized by an EC₅₀ at least 65-fold more selective for the human melanocortin 4 receptor than for the human melanocortin 3 receptor.

In another class of these embodiments, the functional activity of the melanocortin 4 agonist is characterized by an EC₅₀ at least 50-fold more selective for the human melanocortin 4 receptor than for the human melanocortin 5 receptor. In another class of these embodiments, the functional activity of the melanocortin 4 agonist is characterized by an EC₅₀ at least 90-fold more selective for the human melanocortin 4 receptor than for the human melanocortin 5 receptor.

In another embodiment of the present invention, the invention is directed to a method of reducing alcohol consumption comprising administering to a subject a therapeutically effective amount of a non-peptidyl melanocortin 4 receptor agonist, or a pharmaceutically acceptable salt thereof, wherein the binding affinity index of the melanocortin 4 receptor agonist is characterized by an IC₅₀ value at least 35-fold more selective for the human melanocortin 4 receptor than for the human melanocortin 1 receptor, the human melanocortin 3 receptor and the human melanocortin 5 receptor.

In a class of this embodiment, the binding affinity index of the melanocortin 4 agonist is characterized by an IC₅₀ value at least 125-fold more selective for the human melanocortin 4 receptor than for the human melanocortin 1 receptor.

In another class of this embodiment, the binding affinity index of the melanocortin 4 agonist is characterized by an IC₅₀ value at least 35-fold more selective for the human melanocortin 4 receptor than for the human melanocortin 3 receptor. In another class of this embodiment, the binding affinity index of the melanocortin 4 agonist is characterized by an IC₅₀ value at least 160-fold more selective for the human melanocortin 4 receptor than for the human melanocortin 3 receptor.

In another class of this embodiment, the binding affinity index of the melanocortin 4 agonist is characterized by an IC₅₀ value at least 12-fold more selective for the human melanocortin 4 receptor than for the human melanocortin 5 receptor. In another class of this embodiment, the binding affinity index of the melanocortin 4 agonist is characterized by an IC₅₀ value at least 35-fold more selective for the human melanocortin 4 receptor than for the human melanocortin 5 receptor.

In another embodiment of the present invention, the invention is directed to the use of a therapeutically effective amount of a non-peptidyl melanocortin 4 receptor agonist for the manufacture of a medicament useful to inhibit alcohol consumption in a subject in need of such treatment.

In another embodiment of the present invention, the invention is directed to the use of a therapeutically effective amount of a non-peptidyl melanocortin 4 receptor agonist for the manufacture of a medicament useful to reduce alcohol consumption in a subject in need of such treatment. In a class of this embodiment, the alcohol consumption is voluntary alcohol consumption.

In another embodiment of the present invention, the invention is directed to the use of a therapeutically effective amount of a non-peptidyl melanocortin 4 receptor agonist for the manufacture of a medicament useful to treat or prevent alcoholism in a subject in need of such treatment.

In another embodiment of the present invention, the invention is directed to the use of a therapeutically effective amount of a non-peptidyl melanocortin 4 receptor agonist for the manufacture of a medicament useful to treat or prevent alcohol abuse in a subject in need of such treatment.

In another embodiment of the present invention, the invention is directed to the use of a therapeutically effective amount of a non-peptidyl melanocortin 4 receptor agonist for the manufacture of a medicament useful to treat or prevent alcohol-related disorders in a subject in need of such treatment.

In another embodiment of the present invention, the invention is directed to the use of a therapeutically effective amount of a non-peptidyl melanocortin 4 receptor agonist for the manufacture of a medicament useful to prevent a relapse of alcohol consumption to pre-treatment levels following a period of alcohol deprivation in a subject in need of such treatment.

In another embodiment of the present invention, the invention is directed to the use of a therapeutically effective amount of a non-peptidyl melanocortin 4 receptor agonist for the manufacture of a medicament useful to prevent reducing the amount of alcohol consumed as the result of a relapse of alcohol consumption in a subject in need of such treatment.

Melanocortin 4 receptor agonists useful in the methods and medicaments of the present invention are represented by the compounds of structural Formula I and II: 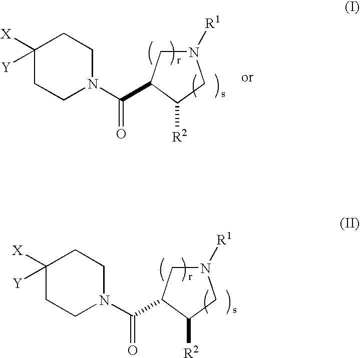
or a pharmaceutically acceptable salt thereof; wherein
X is selected from the group consisting of: C₁₋₈ alkyl, (CH₂)ₙC₃₋₈ cycloalkyl, (CH₂)ₙ-phenyl, (CH₂)ₙ-naphthyl, (CH₂)ₙ-heteroaryl, (CH₂)ₙheterocyclyl, (CH₂)ₙC≡N, (CH₂)ₙCON(R⁵R⁵), (CH₂)ₙCO₂R⁵, (CH₂)ₙCOR⁵, (CH₂)ₙNR⁵C(O)R⁵, (CH₂)ₙNR⁵CO₂R⁵, (CH₂)ₙNR⁵C(O)N(R⁵)₂, (CH₂)ₙNR⁵SO₂R⁵, (CH₂)ₙS(O)ₚR⁵, (CH₂)ₙSO₂N(R⁵)(R⁵), (CH₂)ₙOR⁵, (CH₂)ₙOC(O)R⁵, (CH₂)ₙOC(O)OR⁵, (CH₂)ₙOC(O)N(R⁵)₂, (CH₂)ₙN(R⁵)(R⁵), and (CH₂)ₙNR⁵SO₂N(R⁵)(R⁵), wherein heteroaryl is as defined above; phenyl, naphthyl, and heteroaryl are unsubstituted or substituted with one to three groups independently selected from R⁶; alkyl, cycloalkyl, and heterocyclyl are unsubstituted or substituted with one to three groups independently selected from R⁶ and oxo; and wherein any methylene (CH₂) in X is unsubstituted or substituted with one to two groups independently selected from halogen, hydroxy, and C₁₋₄ alkyl;
Y is selected from the group consisting of: hydrogen, C₁₋₈ alkyl, C₂₋₆ alkenyl, (CH₂)ₙC₃₋₈ cycloalkyl, (CH₂)ₙ-phenyl, (CH₂)ₙ-naphthyl, (CH₂)ₙ-heteroaryl, and (CH₂)ₙ-heterocyclyl,
wherein heteroaryl is as defined above, and phenyl, naphthyl, and heteroaryl are unsubstituted or substituted with one to three groups independently selected from R⁶; alkyl, cycloalkyl, and heterocyclyl are optionally substituted with one to three groups independently selected from R⁶ and oxo; and wherein any methylene (CH₂) in Y is unsubstituted or substituted with one to two groups independently selected from halogen, hydroxy, and C₁₋₄ alkyl;
R¹ is selected from the group consisting of: hydrogen, amidino, C₁₋₄ alkyliminoyl, C₁₋₁₀ alkyl, (CH₂)ₙ—C₃₋₇ cycloalkyl, (CH₂)ₙ-phenyl, (CH₂)ₙ-naphthyl, and (CH₂)ₙ-heteroaryl, wherein heteroaryl is selected from the group consisting of (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³; and alkyl and cycloalkyl are unsubstituted or substituted with one to three groups independently selected from R³ and oxo;
R² is selected from the group consisting of: phenyl, naphthyl, and heteroaryl, wherein heteroaryl is selected from the group consisting of: (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³;
each R³ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃, in which heteroaryl is as defined above; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
each R⁴ is independently selected from the group consisting of: hydrogen, C₁₋₆ alkyl, (CH₂)ₙ phenyl, (CH₂)ₙ-heteroaryl, (CH₂)ₙ-naphthyl, (CH₂)ₙ-heterocyclyl, (CH₂)ₙC₃₋₇ cycloalkyl, and (CH₂)ₙC₃₋₇ bicycloalkyl, wherein alkyl, phenyl, heteroaryl, heterocyclyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl;
each R⁵ is independently selected from the group consisting of: hydrogen, C₁₋₈ alkyl, (CH₂)ₙ-phenyl, (CH₂)ₙ-naphthyl, (CH₂)ₙ-heteroaryl, and (CH₂)ₙC₃₋₇ cycloalkyl, wherein heteroaryl is as defined above; phenyl, naphthyl, and heteroaryl are unsubstituted or substituted with one to three groups independently selected from R³; alkyl and cycloalkyl are unsubstituted or substituted with one to three groups independently selected from R³ and oxo; and wherein any methylene (CH₂) in R⁵ is unsubstituted or substituted with one to two groups independently selected from halogen, hydroxy, and C₁₋₄ alkyl; or two R⁵ groups together with the atom to which they are attached form a 5- to 8-membered mono- or bicyclic ring system optionally containing an additional heteroatom selected from O, S, and NC₁₋₄ alkyl;
each R⁶ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃, in which heteroaryl is as defined above; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R⁶ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
r is 1 or 2;
s is 0, 1, or 2;
n is 0, 1 or 2; and
p is 0, 1, or 2.

In one embodiment of the compounds of structural formula I and II, R¹ is selected from the group consisting of hydrogen, C₁₋₆ alkyl, (CH₂)₀₋₁C₃₋₆ cycloalkyl, and (CH₂)₀₋₁-phenyl; wherein phenyl is unsubstituted or substituted with one to three groups independently selected from R³; and alkyl and cycloalkyl are optionally substituted with one to three groups independently selected from R³ and oxo. In a class of this embodiment, R¹ is tert-butyl.

In a second embodiment of the compounds of structural formula I and II, R² is phenyl or thienyl optionally substituted with one to three groups independently selected from R³. In a class of this embodiment, R² is phenyl optionally substituted with one to three groups independently selected from R³. In another class of this embodiment, R² is phenyl substituted with one to three groups independently selected from R³. In a subclass of this class, R² is phenyl substituted with two groups independently selected from R³. In a subclass of this subclass, R² is phenyl substituted with two halogen groups.

In a third embodiment of the compounds of structural formula I and II, X is selected from the group consisting of: (CH₂)ₙ-phenyl, (CH₂)ₙ-naphthyl, (CH₂)ₙ-heteroaryl, (CH₂)ₙC₃₋₈ cycloalkyl, and (CH₂)ₙ-heterocyclyl, wherein heteroaryl is as defined above, and phenyl, naphthyl, and heteroaryl are optionally substituted with one to three groups independently selected from R⁶; cycloalkyl and heterocyclyl are optionally substituted with one to three groups independently selected from R⁶ and oxo; and wherein any methylene (CH₂) group in X is unsubstituted or substituted with one to two groups independently selected from halogen, hydroxy, and C₁₋₄ alkyl. In a class of this embodiment, X is selected from the group consisting of (CH₂)₀₋₁-phenyl, (CH₂)₀₋₁-heteroaryl, (CH₂)₀₋₁-heterocyclyl; wherein phenyl and heteroaryl are optionally substituted with one to three groups independently selected from R⁶; heterocyclyl is optionally substituted with one to three groups independently selected from R⁶ and oxo; and CH₂ is unsubstituted or substituted with one to two groups independently selected from halogen, hydroxy, and C₁₋₄ alkyl. In a subclass of this class, X is phenyl optionally substituted with one to three groups independently selected from R⁶.

In a fourth embodiment of compounds of formula I and II, Y is hydrogen.

In yet a further embodiment of compounds of structural formula I and II, r is 1 or 2 and s is 1.

A specific melanocortin-4 receptor agonist useful in the methods of the present invention is Compound A:

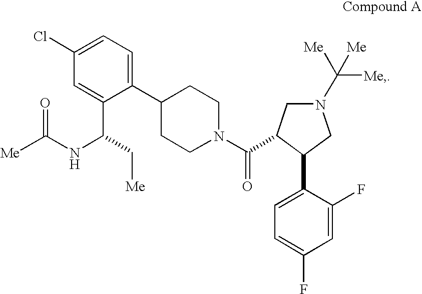

Compound A is a selective agonist for MC4R, with a selective functional activity characterized by an EC₅₀ value for the melanocortin 4 receptor that is at least 120-fold lower than for the melanocortin 1 receptor, at least 700-fold lower than for the melanocortin 2 receptor, at least 65-fold lower than for the melanocortin 3 receptor, and at least 100 fold lower than for the melanocortin 5 receptor.

Other melanocortin-4 receptor agonists useful in the methods of the present invention include, but are not limited to, the following: 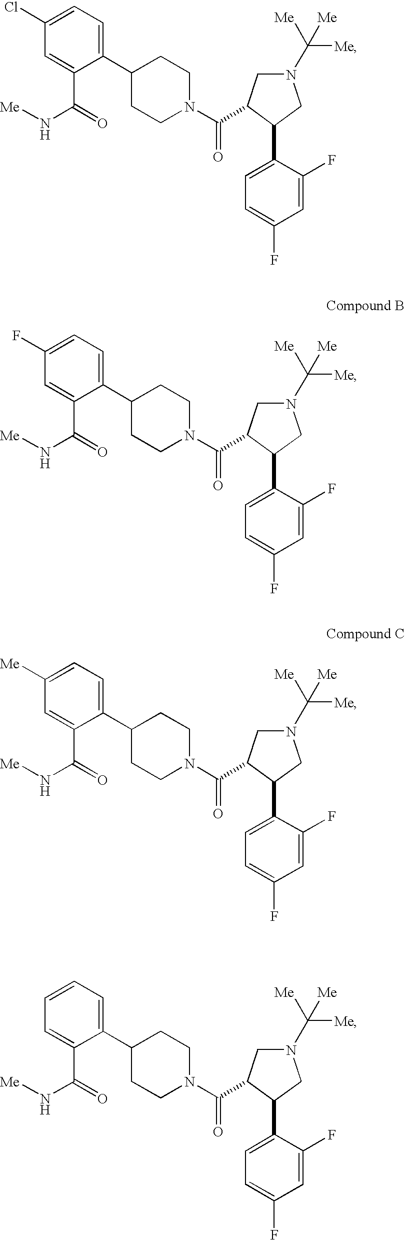 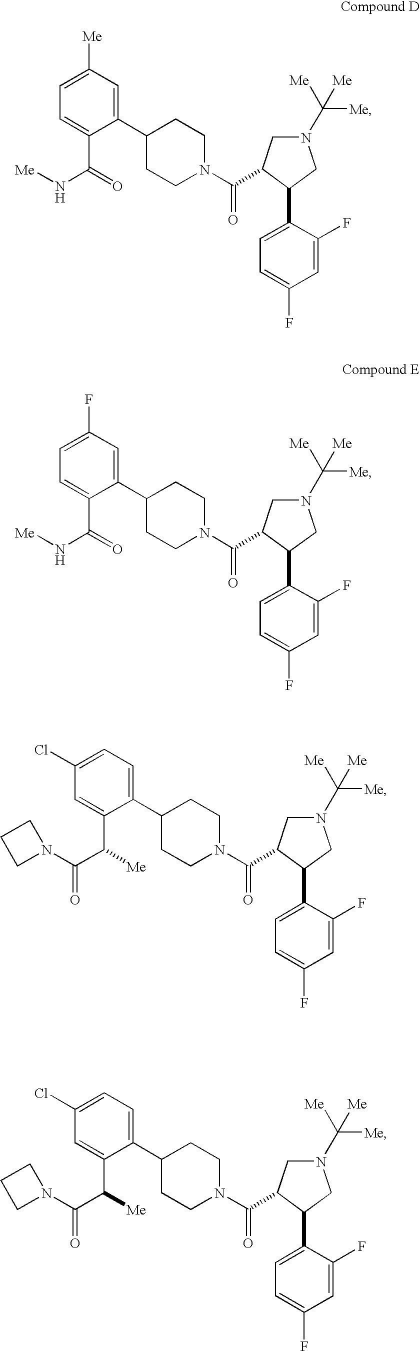 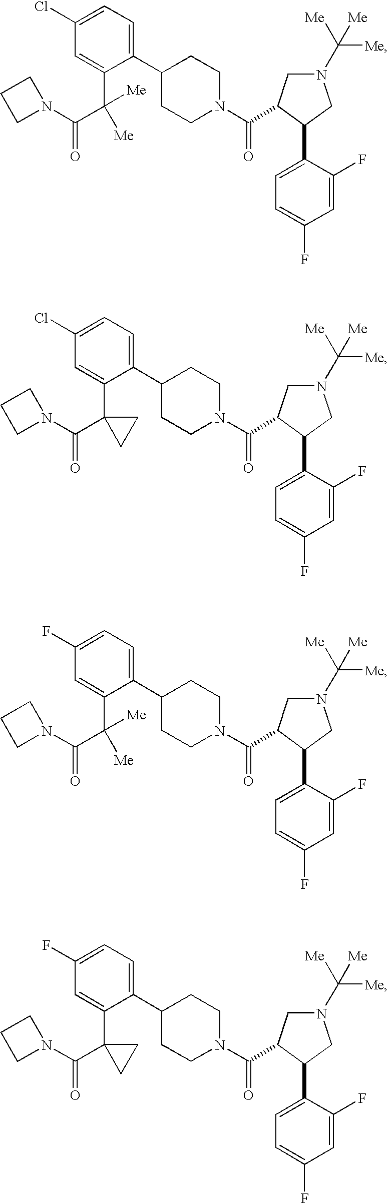 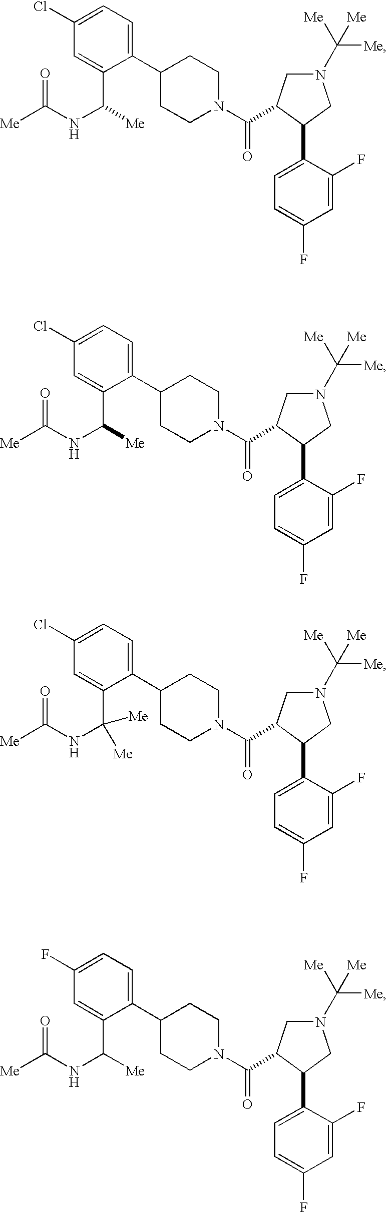 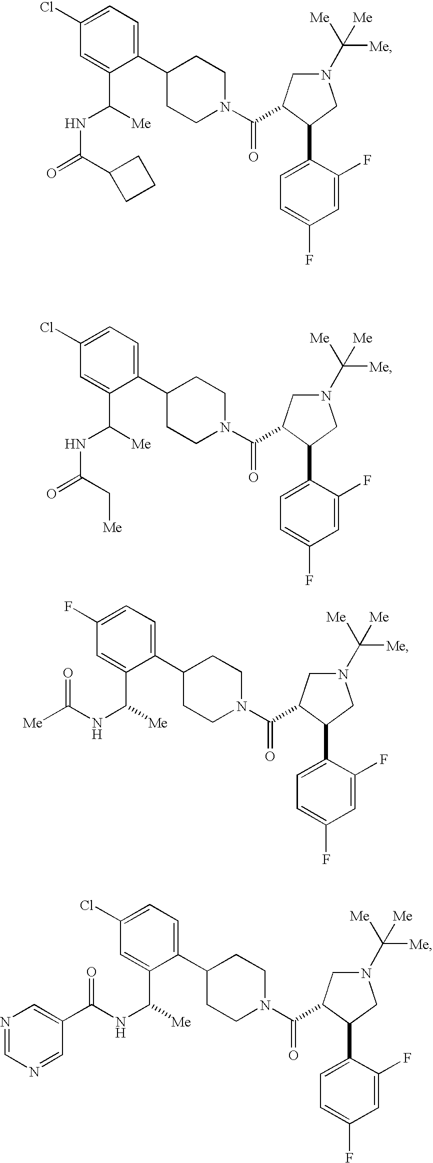 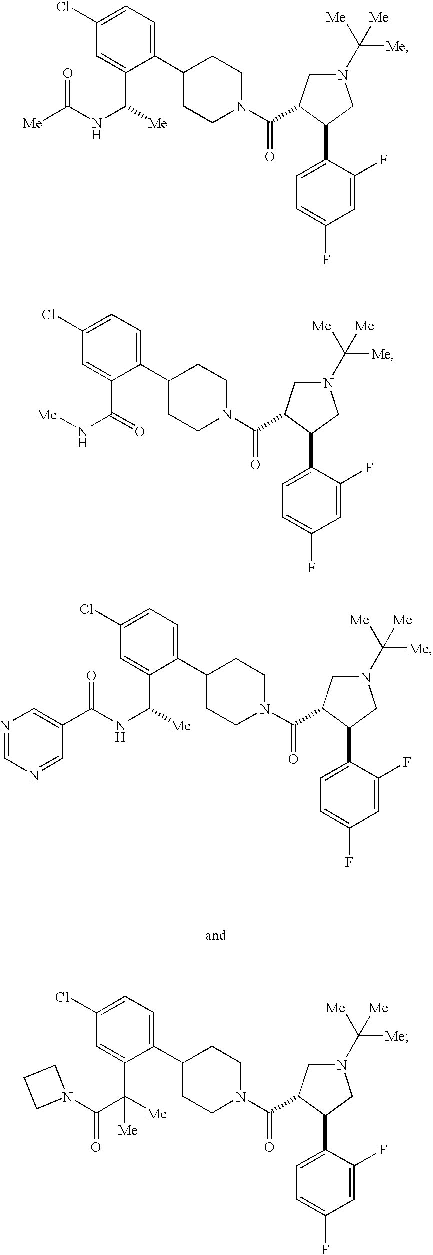
or a pharmaceutically acceptable salt thereof.

The melanocortin 4 receptor agonists of Formula I and II, including Compound A, and their preparation are disclosed in US2003/0225060, which is hereby incorporated by reference in its entirety, WO 02/068387, and WO 02/068388.

The above compounds are only illustrative of the non-peptidyl MC4R agonists that can be used in the compositions of the present invention. As this listing of compounds is not meant to be comprehensive, the methods of the present invention may employ any non-peptidyl MC4R agonists, including the MC4R agonists of Formulas I, II, and Compound A, and are not limited to any particular structural class of compounds.

Other melanocortin 4 receptor agonists useful in the present invention, include but are not limited to those disclosed in U.S. Pat. No. 6,294,534, U.S. Pat. Nos. 6,350,760, 6,376,509, 6,410,548, 6,458,790, U.S. Pat. No. 6,472,398, U.S. Pat. No. 5,837,521, U.S. Pat. No. 6,699,873, which are hereby incorporated by reference in their entirety; in US Patent Application Publication Nos. US 2002/0004512, US2002/0019523, US2002/0137664, US2003/0236262, US2003/0225060, US2003/0092732, US2003/109556, US 2002/0177151, US 2002/187932, US 2003/0113263, which are hereby incorporated by reference in their entirety; and in WO 99/64002, WO 00/74679, WO 02/15909, WO 01/70708, WO 01/70337, WO 01/91752, WO 02/068387, WO 02/068388, WO 02/067869, WO 03/007949, WO 2004/024720, WO 2004/037797, WO 04/078717, WO 04/087159, WO 04/089307, WO 05/009950, WO 01/58891, WO 02/070511, WO 02/079146, WO 03/009847, WO 03/057671, WO 03/068738, WO 03/092690, WO 02/059095, WO 02/059107, WO 02/059108, WO 02/059117, WO 02/085925, WO 03/004480, WO 03/009850, WO 03/013571, WO 03/031410, WO 03/053927, WO 03/061660, WO 03/066597, WO 03/094918, WO 03/099818, WO 04/037797, WO 04/048345, WO 04/058735, WO 02/018327, WO 02/080896, WO 02/081443, WO 03/066587, WO 03/066597, WO 03/099818, WO 02/062766, WO 03/000663, WO 03/000666, WO 03/003977, WO 03/040107, WO 03/040117, WO 03/040118, WO 03/013509, WO 03/057671, WO 02/079753, WO 02//092566, WO 03/-093234, WO 03/095474 and WO 03/104761.

One of ordinary skill in the art can readily identify melanocortin 4 receptor agonist compounds useful in the compositions and methods of the present invention using the methods described in Example 1. MC4R agonists which are useful in the present invention generally have an IC₅₀ less than 100 nM in the MC4R agonist binding assay and an EC₅₀ less than 100 nM in the functional assay described in Example 2. Particularly useful in the present invention are MC4R agonists with an IC₅₀ less than 45 nM or an EC₅₀ less than 15 nM. More particularly useful in the present in invention are MC4R agonists with an EC₅₀ less than 15 nM and an IC₅₀ less than 45 nM.

Throughout the instant application, the following terms have the indicated meanings:

The alkyl groups specified above are intended to include those alkyl groups of the designated length in either a straight or branched configuration. Exemplary of such alkyl groups are methyl, ethyl, propyl, isopropyl, butyl, sec-butyl, tertiary butyl, pentyl, isopentyl, hexyl, isohexyl, and the like.

The term “halogen” is intended to include the halogen atoms fluorine, chlorine, bromine and iodine.

The term “C₁₋₄ alkyliminoyl” means C₁₋₃C(═NH)—.

The term “aryl” includes phenyl and naphthyl.

The term “heteroaryl” includes mono- and bicyclic aromatic rings containing from 1 to 4 heteroatoms selected from nitrogen, oxygen and sulfur. “5- or 6-Membered heteroaryl” represents a monocyclic heteroaromatic ring; examples thereof include thiazole, oxazole, thiophene, furan, pyrrole, imidazole, isoxazole, pyrazole, triazole, thiadiazole, tetrazole, oxadiazole, pyridine, pyridazine, pyrimidine, pyrazine, and the like. Bicyclic heteroaromatic rings include, but are not limited to, benzothiadiazole, indole, benzothiophene, benzofuran, benzimidazole, benzisoxazole, benzothiazole, quinoline, benzotriazole, benzoxazole, isoquinoline, purine, furopyridine and thienopyridine.

The term “5- or 6-membered carbocyclyl” is intended to include non-aromatic rings containing only carbon atoms such as cyclopentyl and cyclohexyl.

The term “5 and 6-membered heterocyclyl” is intended to include non-aromatic heterocycles containing one to four heteroatoms selected from nitrogen, oxygen and sulfur. Examples of a 5 or 6-membered heterocyclyl include piperidine, morpholine, thiamorpholine, pyrrolidine, imidazolidine, tetrahydrofuran, piperazine, and the like.

Certain of the above defined terms may occur more than once in the above formula and upon such occurrence each term shall be defined independently of the other; thus for example, NR⁴R⁴ may represent NH₂, NHCH₃, N(CH₃)CH₂CH₃, and the like.

It will be understood that, as used herein, references to MC4R agonists, including MC4R agonists of Formulas I and II, and compound A, are meant to also include the pharmaceutically acceptable salts and esters thereof.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, lithium, magnesium, potassium, and sodium salts. Salts derived from pharmaceutically acceptable organic nontoxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like. Particularly preferred are citric, fumaric, hydrobromic, hydrochloric, maleic, phosphoric, sulfuric, and tartaric acids. It will be understood that, as used herein, references to the compounds of Formula I, formula II, compound A, and other melanocortin 4 receptor agonists, are meant to also include the pharmaceutically acceptable salts, such as the hydrochloride salts.

The compounds useful in the methods of the present invention include stereoisomers, such as optical isomers, diastereomers and geometerical isomers, or tautomers depending on the mode of substitution. The compounds may contain one or more chiral centers and occur as racemates, racemic mixtures and as individual diastereomers, enantiomeric mixtures or single enantiomers, keto-enol tautomers, or E and Z olefinic geometric isomers, with all isomeric forms being included in the present invention. The present invention is meant to comprehend all such isomeric forms of the compounds in the compositions of the present invention, and their mixtures. Therefore, where a compound is chiral, the separate enantiomers, substantially free of the other, are included within the scope of the invention; further included are all mixtures of the two enantiomers. Also included within the scope of the invention are polymorphs, hydrates and solvates of the compounds of the instant invention.

The present invention includes within its scope prodrugs of the compounds in the compositions of this invention. In general, such prodrugs will be functional derivatives of the compounds in these compositions which are readily convertible in vivo into the required compound. Thus, in the methods of treatment of the present invention, the term “administering” shall encompass the treatment of alcoholism, alcohol abuse, alcohol consumption and alcohol related disorders with the compounds specifically disclosed as elements of the composition or with compounds which may not be specifically disclosed, but which convert to the specified compounds in vivo after administration to the patient. Conventional procedures for the selection and preparation of suitable prodrug derivatives are described, for example, in “Design of Prodrugs,” ed. H. Bundgaard, Elsevier, 1985.

Utility

The compounds of the present invention are useful to inhibit or reduce voluntary alcohol consumption, and for the treatment or prevention of alcoholism, alcohol abuse, and alcohol-related disorders.

Alcoholism, also known as alcohol dependence, is a disease that is characterized by abnormal alcohol seeking behavior that leads to impaired control over drinking. Alcoholism may include some or all of the following symptoms: narrowing of drinking repertoire (drinking only one brand or type of alcoholic beverage); craving (a strong need or urge to drink), loss of control (not being able to stop drinking once drinking has begun), drink seeking behavior (attending only social events that include drinking); physical dependence (withdrawal symptoms, such as nausea, sweating, shakiness, and anxiety after cessation of drinking), drinking to relieve or avoid withdrawal symptoms; and tolerance (the need to drink greater amounts of alcohol to achieve previous effects); subjective awareness of the compulsion to drink or craving for alcohol; and relapse (a return to drinking after a period of abstinence).

Alcohol abuse is a pattern of drinking that results in one or more of the following situations within a 12 month period: failure to fulfill major work, school or home responsibilities; drinking in situations that are physically dangerous, such as while driving a car or operating machinery; having recurring alcohol related legal problems, such as being arrested for driving under the influence of alcohol, or physically hurting someone while drunk; and continued drinking despite ongoing relationship problems that are caused or worsened by the drinking. Harmful alcohol use implies alcohol use that causes health consequences, such as physical or mental damage. Alcohol related disorders include, but are not limited to, disorders resulting from alcohol dependence, alcohol abuse and alcohol consumption. Alcohol related disorders include, but are not limited to: liver disease, such as hepatitis, inflammation of the liver, and alcoholic cirrhosis; heart disease; high blood pressure; stroke; certain forms of cancer, such as esophageal, mouth, throat, voice box, breast, colon and rectal cancer; pancreatitis; alcoholic dementia, Wernicke-Korsakoff syndrome, brain damage, slow bone healing; impaired wound healing; diminished immune defenses; and death. Alcohol misuse has also been found to predispose the subject to osteoporosis, slow bone healing, impaired wound healing, inhibited osteoblastic function and diminished immune defenses. Alcohol intoxication increases the risk of further accidents, and decreases the pain inhibition that would make a normal patient more careful. Alcohol dependence also leads to altered cognitive and emotional functions, and thought processes, such as impaired judgment, feelings of incompetency, low self-esteem, despair in relationships, depression, and feelings of failure.

“Treatment” (of alcoholism or alcohol abuse) refers to the administration of the compounds or combinations of the present invention to reduce or inhibit the consumption of alcohol in a subject. One outcome of treatment may be reducing the consumption of alcohol in a subject relative to the subject's alcohol consumption prior to treatment. Another outcome of treatment may be inhibiting consumption of alcohol in a subject. Another outcome of treatment may be decreasing the occurrence of alcohol intake in a subject. Another outcome of treatment may be decreasing the severity of alcohol intake, such as decreasing the amount of alcohol consumed, in a subject. Another outcome of treatment may be to administer the compounds or combinations of the present invention to reduce or inhibit the consumption of alcohol in a subject in need thereof.

The term “inhibit” alcohol consumption means to stop alcohol consumption in a subject. One outcome of inhibition may be to stop alcohol consumption in a subject in need thereof.

The term “reduce” alcohol consumption means to decrease the amount of alcohol consumed by a subject relative to the amount of alcohol consumed prior to the start of treatment. In one embodiment the amount of alcohol consumed by a subject is decreased by at least 10% relative to the amount of alcohol consumed prior to the start of treatment. In another embodiment, the amount of alcohol consumed by a subject is decreased by at least 25% relative to the amount of alcohol consumed prior to the start of treatment. In another embodiment, the amount of alcohol consumed by a subject is decreased by at least 67% relative to the amount of alcohol consumed prior to the start of treatment. In yet another embodiment, the amount of alcohol consumed by a subject is decreased by at least 86% relative to the amount of alcohol consumed prior to the start of treatment.

“Prevention” (of alcoholism) refers to the administration of the compounds or combinations of the present invention to prevent alcohol intake, alcohol consumption, alcohol abuse, alcoholism or developing an alcohol-related disorder in a subject by administration prior to the start of alcohol consumption. One outcome of prevention may be to prevent alcohol intake in a subject by administration prior to the start of alcohol consumption. Another outcome of prevention may be to prevent alcohol abuse in a subject. Another outcome of prevention may be to prevent alcoholism in a subject. Another outcome of prevention may be to prevent the development of an alcohol-related disorder in a subject. Another outcome of prevention may be preventing alcohol consumption from occurring if the treatment is administered prior to the onset of alcohol consumption in a subject. Another outcome of prevention may be to prolong resistance to alcohol consumption in a subject. Another outcome of prevention may be to administer the compounds or combinations of the present invention to prevent alcohol intake in a subject at risk of alcohol consumption, alcohol abuse, alcoholism or developing an alcohol-related disorder in a subject.

Moreover, if treatment is commenced in a subject already consuming alcohol, such treatment may prevent the occurrence, progression or severity of alcohol-related disorders, such as, but not limited to, liver disease; hepatitis; inflammation of the liver; alcoholic cirrhosis; heart disease; high blood pressure; stroke; esophageal cancer, mouth cancer; throat cancer; voice box cancer; breast cancer; colon cancer; rectal cancer; pancreatitis; alcoholic dementia; Wernicke-Korsakoff syndrome; brain damage; osteoporosis; slow bone healing; impaired wound healing; diminished immune defenses; depression; and death.

The term “alcohol” is understood to mean ethanol.

The term “selective” means having an activation preference for a specific receptor over other receptors which can be quantified based upon whole cell, tissue, or organism assays which demonstrate receptor activity, such as the cAMP Functional Assay and the Binding Affinity Assay. The compounds of the present invention interact preferentially (i.e. selectively) with the MC-4 receptor relative to the other melanocortin receptors. Selectivity for the MC-4 receptor is beneficial for compounds administered to humans or mammals, to minimize the number of side effects associated with their administration. MC-4 selectivity of a compound over another MC receptor is defined herein as the EC₅₀, or IC₅₀, of the compound at the MC receptor being referenced over the EC₅₀, or IC₅₀, of the compound for the MC-4 receptor. As used herein, unless indicated otherwise, use of the term “selective over the other MC receptors” means selective with respect to the other melanocortin receptors, including the MC-1, MC-2, MC-3 and MC-5 receptors. For example, a compound having an EC₅₀ of 8 nM at the MC-4 receptor and an EC₅₀ of >80 nM at the MC-1, MC-2 MC-3, and MC-5 receptors has a selectivity ratio for the MC-4 receptor over the other MC receptors of at least 1:10. Additionally, the term “selective” may also refer to one of the MC-1, MC-2, MC-3 or MC-5 receptors individually. For example, a compound having an EC₅₀ of 8 nM at the MC-4 receptor and an EC₅₀ of 80 nM at the MC-1 receptor has a selectivity ratio for the MC-4 receptor over the MC-1 receptor of 1:10. Such a compound is selective over the MC-1 receptor regardless of its EC₅₀ value for the MC-2R or MC-5R. For example, the selectivity of a compound for the MC-4R over the MC-1R is defined as: MC-4R functional selectivity, EC₅₀=[EC₅₀ MC-1R]/[EC₅₀ MC-4R], or MC-4R binding selectivity, IC₅₀=[IC₅₀ MC-1R]/[IC₅₀ MC-4R]. A compound is defined herein as being “selective” for the MC-4 receptor when the above mentioned ratio is at least 10, preferably at least 65, and more preferably at least about 100.

The terms “administration of” and or “administering” a compound should be understood to mean providing a compound of the invention or a prodrug of a compound of the invention to a subject. The instant pharmaceutical composition includes administration of a single pharmaceutical dosage formulation which contains the melanocortin 4 receptor agonist.

The term “subject”, as used herein refers to an animal, preferably a mammal, more preferably a human. In one embodiment of the present invention, the term “subject” refers to a human that is or has been the object of treatment, observation or experiment. In another embodiment of the present invention, the term “subject” refers to a “subject in need thereof”. In a class of this embodiment, the “subject in need thereof” refers to a subject who is in need of treatment or prophylaxis as determined by a researcher, veterinarian, medical doctor or other clinician. In another embodiment, the “subject in need thereof” is a human that is alcohol dependent. In another embodiment, the “subject in need thereof” is a human that is an alcoholic. In another embodiment, the “subject in need thereof” is a human that abuses alcohol. In another embodiment, the “subject in need thereof” is a human that consumes alcohol. In another embodiment, the “subject in need thereof” has an alcohol-related disorder. In another embodiment, the “subject in need thereof” is a subject experiencing the alcohol deprivation effect. In yet another embodiment of the present invention, the term “subject” refers to a “subject at risk thereof”. In a class of this embodiment, the “subject at risk thereof” is a subject at risk of developing alcoholism. In another class of this embodiment, the “subject at risk thereof” is a subject at risk of developing an alcohol-related disorder. In another embodiment, the “subject in need thereof” is at risk of experiencing the alcohol deprivation effect. An embodiment of the term “mammal in need thereof” is a “human in need thereof,” said human being either male or female.

The term “a subject currently consuming alcohol” as used herein refers to a subject that has not undergone alcohol detoxification and has not achieved alcohol abstinence at the time of treatment initiation.

The administration of the composition of the present invention in order to practice the present methods of therapy is carried out by administering a therapeutically effective amount of the compounds in the composition to a subject in need of such treatment or prophylaxis. The need for a prophylactic administration according to the methods of the present invention is determined via the use of well known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgment.

The term “therapeutically effective amount” as used herein means the amount of the active compounds in the composition that will elicit the biological or medical response in a tissue, system, subject, or human that is being sought by the researcher, veterinarian, medical doctor or other clinician, which includes alleviation of the symptoms of the disorder being treated. Disorders being treated include, but are not limited to, alcohol dependence, alcoholism, or alcohol abuse, or alcohol consumption or an alcohol-related disorder in subjects in need thereof. The novel methods of treatment of this invention are for disorders known to those skilled in the art.

The term “prophylactically effective amount” as used herein means the amount of the active compounds that will elicit the biological or medical response in a tissue, system, subject, or human that is being sought by the researcher, veterinarian, medical doctor or other clinician, to prevent the onset of alcohol dependence, alcoholism, or alcohol abuse, or alcohol consumption or an alcohol-related disorder in subjects as risk for alcohol dependence, alcoholism, alcohol abuse, alcohol consumption or an alcohol-related disorder.

The term “alcohol deprivation effect” as used herein means the transient increase in oral ethanol self administration following forced deprivation of alcohol consumption, or abstinence from alcohol consumption after a period of alcohol consumption, and is characterized by loss of control over intake.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

By a melanocortin receptor “agonist” is meant an endogenous or drug substance or compound that can interact with a melanocortin receptor and initiate a pharmacological response characteristic of the melanocortin receptor. By a melanocortin receptor “antagonist” is meant a drug or a compound that opposes the melanocortin receptor-associated responses normally induced by another bioactive agent. The “agonistic” properties of the compounds of the present invention were measured in the functional assay described below. The functional assay discriminates a melanocortin receptor agonist from a melanocortin receptor antagonist.

By “binding affinity” and “binding affinity index” is meant the ability of a compound/drug to bind to its biological target, in the present instance, the ability of a melanocortin 4 receptor agonist, including compounds of structural formula I and II, to bind to a melanocortin receptor. Binding affinities for the compounds of the present invention were measured in the binding assay described below and are expressed as IC₅₀ values.

“Efficacy” describes the relative intensity with which agonists vary in the response they produce even when they occupy the same number of receptors and with the same affinity. Efficacy is the property that enables drugs to produce responses. Properties of compounds/drugs can be categorized into two groups, those which cause them to associate with the receptors (binding affinity) and those that produce a stimulus (efficacy). The term “efficacy” is used to characterize the level of maximal responses induced by agonists. Not all agonists of a receptor are capable of inducing identical levels of maximal responses. Maximal response depends on the efficiency of receptor coupling, that is, from the cascade of events, which, from the binding of the drug to the receptor, leads to the desired biological effect.

The functional activities expressed as EC₅₀'s and the “agonist efficacy” for the compounds of the present invention at a particular concentration were measured in the functional assay described below.

The magnitude of prophylactic or therapeutic dose of the active ingredient (the MC4R agonist) will, of course, vary with the nature of the severity of the condition to be treated and with the particular compound and its route of administration. It will also vary according to the age, weight and response of the individual patient. In general, the daily dose range of each compound lies within the range of from about 0.001 mg/kg to about 1000 mg/kg; more specifically from about 0.001 mg/kg to about 100 mg/kg body weight of a subject per day in single or divided doses. On the other hand, it may be necessary to use dosages outside these limits in some cases.

For use where a composition for intravenous administration is employed, a suitable dosage range is from about 0.001 mg/kg to about 1000 mg/kg; more specifically from 0.001 mg/kg to about 100 mg/kg of each compound in the composition per day.

In the case where an oral composition is employed, a suitable dosage range is, e.g. from about 0.001 mg/kg to about 1000 mg/kg of each compound in the composition per day; more specifically from about 0.001 mg/kg to about 100 mg/kg per day. For oral administration, the compositions are provided in the form of tablets containing from 0.01 mg to 1,000 mg; more specifically 0.01, 0.05, 0.1, 0.2, 0.5, 1.0, 2.5, 5, 10, 15, 20, 25, 30, 40, 50, 75, 100, 125, 150, 175, 200, 225, 250, 500, 650, 750, 850 and 1,000 milligrams of each active ingredient for the symptomatic adjustment of the dosage to the subject to be treated. This dosage regimen may be adjusted to provide the optimal therapeutic response.

The compounds of this invention can be administered to humans in the dosage ranges specific for each compound. In general, for treating alcoholism, alcohol abuse, alcohol consumption and/or an alcohol related disorder, the MC4R agonist is administered at a daily dosage of from about 0.001 mg/kg to about 1000 mg/kg of body weight orally. More specifically, when treating alcoholism, alcohol abuse, alcohol consumption and/or an alcohol related disorder, generally satisfactory results may be obtained when a MC4R agonist, such as a MC4R agonist of Formula I, Formula II and Compound A, or a pharmaceutically acceptable salt or ester thereof, is administered at a daily oral dosage of from about 0.001 mg/kg to about 1000 mg/kg; more specifically from about 0.001 mg/kg to about 100 mg/kg of body weight, given in a single dose or in divided doses two to six times a day, or in sustained release form. In the case of a 70 kg adult human, the total daily dose will generally be from about 0.07 milligrams to about 3500 milligrams.

The effective dosage of the active ingredient employed in the composition may vary depending on the particular compound employed, the mode of administration, the condition being treated and the severity of the condition being treated. Thus, the dosage regimen utilizing the compositions of the present invention is selected in accordance with a variety of factors including type, species, age, general health, body weight, diet, sex and medical condition of the subject; the severity of the condition to be treated; the renal and hepatic function of the patient; the drug combination; and the particular compound employed and its route of administration. A physician, clinician or veterinarian of ordinary skill can readily determine and prescribe the effective amount of the drug required to prevent, counter or arrest the progress of the condition.

Combination Therapy

The melanocortin 4 receptor agonists useful in the methods of the present invention may be used in combination with other drugs that are used in the treatment or prevention of alcoholism, alcohol abuse, alcohol consumption and/or an alcohol related disorder. Such other drugs may be administered, by a route and in an amount commonly used therefor, contemporaneously or sequentially with a melanocortin 4 receptor agonist. When a melanocortin 4 receptor agonist is used contemporaneously with one or more other drugs, a pharmaceutical composition containing such other drugs in addition to the melanocortin 4 receptor agonist is preferred. Accordingly, the pharmaceutical compositions useful in the methods of the present invention include those that also contain one or more other active ingredients, in addition to a melanocortin 4 receptor agonist.

Examples of other active ingredients that may be combined with the melanocortin 4 receptor agonists useful in the present invention for the treatment or prevention of alcoholism, alcohol abuse, alcohol consumption and/or an alcohol related disorder, either administered separately or in the same pharmaceutical compositions, include, but are not limited to:

(a) cannabinoid CB-1 antagonists/inverse agonists, such as rimonabant;

(b) bupropion;

(c) opioid antagonists, such as naloxone, naltrexone, and nalmefene;

(d) inhibitors of acetaldehye metabolism, such as disulfuram;

(e) dopamine D3 antagonists/inverse agonists; and

(f) GABA agonists such as acamprosate.

Additionally, the use of combination therapy in the methods of the present invention, comprising administering a therapeutically effective amount of a melanocortin 4 receptor agonist with a therapeutically effective or subtherapeutically effective amount, of a medication approved to inhibit alcohol consumption, that acts via a different complementary mechanism, will result in improved efficacy.

Pharmaceutical Compositions

Another aspect of the present invention provides pharmaceutical compositions useful for the methods of the present invention comprising a pharmaceutical carrier and a therapeutically effective amount of a melanocortin-4 receptor agonist and each additional compound in the composition of the present invention. The term “composition”, as in pharmaceutical composition, is intended to encompass a product comprising the active ingredient(s), and the inert ingredient(s), such as pharmaceutically acceptable excipients, that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a melanocortin 4 receptor agonist, additional active ingredient(s), and/or pharmaceutically acceptable excipients and carriers.

Any suitable route of administration may be employed for providing a subject, especially a human, with an effective dosage of a composition of the present invention. For example, oral delivery, rectal delivery, topical delivery, parenteral delivery, ocular delivery, pulmonary delivery, nasal delivery, delivery to the central nervous system, in particular the brain, and the like may be employed. Dosage forms include tablets, troches, dispersions, suspensions, solutions, capsules, creams, ointments, aerosols, and the like.

The pharmaceutical compositions of the present invention comprise a MC4R agonist, as active ingredient or a pharmaceutically acceptable salt or ester thereof, and may also contain a pharmaceutically acceptable carrier and optionally other therapeutic ingredients. By “pharmaceutically acceptable” it is meant the carrier, diluent or excipient must be compatible with the other ingredients of the formulation and not deleterious to the recipient thereof. In particular, the term “pharmaceutically acceptable salts” refers to salts prepared from pharmaceutically acceptable non-toxic bases or acids including inorganic bases or acids and organic bases or acids.

The compositions include compounds suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. These compositions may be conveniently presented in unit dosage form and prepared by any of the methods well-known in the art of pharmacy.

For administration by inhalation, the compositions of the present invention are conveniently delivered in the form of an aerosol spray presentation from pressurized packs or nebulizers. The compositions may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of the instant composition in suitable propellants, such as fluorocarbons or hydrocarbons and dry powder inhalation (DPI) aerosol, which may be formulated as a dry powder of the composition with or without additional excipients. Suitable topical formulations of the compositions of the present invention include transdermal devices, aerosols, creams, solutions, ointments, gels, lotions, dusting powders, and the like. The topical pharmaceutical compositions containing the compositions of the present invention ordinarily include about 0.005% to 5% by weight of the active compounds in admixture with a pharmaceutically acceptable vehicle. Transdermal skin patches useful for administering the compositions of the present invention include those well known to those of ordinary skill in that art. To be administered in the form of a transdermal delivery system, the dosage administration will, of course be continuous rather than intermittent throughout the dosage regimen. The compositions of the present invention can also be administered in the form of liposome delivery systems, such as small unilamellar vesicles, large unilamellar vesicles and multilamellar vesicles. Liposomes can be formed from a variety of phospholipids, such as cholesterol, sterylamine or phosphatidylcholines. Compositions of the present invention may also be delivered by the use of monoclonal antibodies as individual carriers to which the compound molecules are coupled. The compounds in these compositions may also be coupled with soluble polymers as targetable drug carriers. Such polymers can include polyvinylpyrrolidone, pyran copolymer, polyhydroxypropyl-methacrylamide phenol, polyhydroxyethylasparamidepheon, or polyethyleneoxidepolylysine substituted with palmitoyl residues. Furthermore, the compositions of the present invention may be coupled to a class of biodegradable polymers useful in achieving controlled release of a drug, for example, polylactic acid, polyepsilon caprolactone, polyhydroxybutyric acid, polyorthoesters, polyacetals, polydihydropyrans, polycyanoacrylates and cross-linked or amphipathic block copolymers of hydrogels.

In practical use, each compound in the compositions of the present invention (e.g. MC4R agonist) can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules, pellet, powder and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the composition may also be administered by controlled release means and/or delivery devices such as those described in U.S. Pat. Nos. 3,845,770; 3,916,899; 3,536,809; 3,598,123; 3,630,200 and 4,008,719, which are hereby incorporated by reference in their entirety.

For example, for oral administration in the form of a tablet, capsule, pellet, or powder, the active ingredient can be combined with an oral, non-toxic, pharmaceutically acceptable inert carrier such as lactose, starch, sucrose, glucose, methyl cellulose, magnesium stearate, mannitol, sorbitol, croscarmellose sodium and the like; for oral administration in liquid form, e.g., elixirs, syrups, slurries, emulsions, suspensions, solutions, and effervescent compositions, the oral drug components can be combined with any oral, non-toxic, pharmaceutically acceptable inert carrier such as ethanol, glycerol, water, oils and the like. Moreover, when desired or necessary, suitable binders, lubricants, disintegrating agents, buffers, coatings, and coloring agents can also be incorporated. Suitable binders can include starch, gelatin, natural sugars such a glucose, anhydrous lactose, free-flow lactose, beta-lactose, and corn sweeteners, natural and synthetic gums, such as acacia, guar, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like. Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. When a dosage unit form is a capsule, it may contain, in addition to materials of the above type, a liquid carrier such as a fatty oil. The active compounds can also be administered intranasally as, for example, liquid drops or spray.

Desirably, each tablet contains from 0.01 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 600, 750, 850 and 1,000 milligrams of each active ingredient in the composition of the present invention (e.g. MC4R agonist) for the symptomatic adjustment of the dosage to the subject to be treated; and each cachet or capsule contains from about 0.01 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 600, 750, 850 and 1,000 milligrams of each active in the composition of the present invention (e.g. MC4R agonist) for the symptomatic adjustment of the dosage to the subject to be treated.

Exemplifying the invention is a pharmaceutical composition comprising a MC4R agonist described above and a pharmaceutically acceptable carrier. Also exemplifying the invention is a pharmaceutical composition made by combining any of the MC4R agonists described above and a pharmaceutically acceptable carrier. An illustration of the invention is a process for making a pharmaceutical composition comprising combining any of the MC4R agonists described above and a pharmaceutically acceptable carrier.

The dose may be administered in a single daily dose or the total daily dosage may be administered in divided doses of two to six times daily. Furthermore, based on the properties of the individual compound selected for administration, the dose may be administered less frequently, e.g., weekly, twice weekly, monthly, etc. The unit dosage will, of course, be correspondingly larger for the less frequent administration.

When administered via intranasal routes, transdermal routes, by rectal or vaginal suppositories, or through a continual intravenous solution, the dosage administration will, of course, be continuous rather than intermittent throughout the dosage regimen.

The following are examples of representative pharmaceutical dosage forms for the compositions of the present invention:

| Tablet | mg/tablet | |
|---|---|---|
| Compound A | 25 | |
| Microcrystalline Cellulose | 50.5 | |
| Lactose | 111.5 | |
| Croscarmellose Sodium | 5.0 | |
| Hydroxypropylcellulose | 6.0 | |
| Sodium Dodecyl Sulfate | 1.0 | |
| Magnesium Stearate | 1.0 | |
| | 200 | |
| Capsule | mg/capsule | |
| Compound A | 100 | |
| Lactose | 80 | |
| Sodium Dodecyl Sulfate | 20 | |
| | 200 | |
| Aerosol | Per canister | |
| Compound A | 13 | mg |
| Lecithin, NF Liq. Conc. | 1.2 | mg |
| Trichlorofluoromethane, NF | 4.65 | g |
| Dichlorodifluoromethane, NF | 12.15 | g |
| | 30 | |

Combination Therapy

It will be understood that the scope of compositions of the compounds of this invention with other agents useful to inhibit or reduce alcohol consumption and for treating or preventing alcoholism, alcohol abuse, and alcohol related conditions includes in principle any combination with any pharmaceutical composition useful to inhibit or reduce alcohol consumption and for treating alcoholism, alcohol abuse and alcohol related disorders.

In order to illustrate the invention, the following examples are included. These examples do not limit the invention. They are only meant to suggest a method of reducing the invention to practice. Those skilled in the art may find other methods of practicing the invention which are readily apparent to them. However, those methods are also deemed to be within the scope of this invention.

### EXAMPLE 1

### Binding Assay

The membrane binding assay was used to identify competitive inhibitors of ¹²⁵I-NDP-alpha-MSH binding to cloned human MCRs expressed in mouse L- or Chinese hamster ovary (CHO)-cells.

Cell lines expressing melanocortin receptors were grown in T-180 flasks containing selective medium of the composition: 1 L Dulbecco's modified Eagles Medium (DMEM) with 4.5 g L-glucose, 25 mM Hepes, without sodium pyruvate, (Gibco/BR1); 100 ml 10% heat-inactivated fetal bovine serum (Sigma); 10 mL 10,000 unit/mL penicillin & 10,000 μg/mL streptomycin (Gibco/BR1); 10 ml 200 mM L-glutamine (Gibco/BR1); 1 mg/mL geneticin (G418) (Gibco/BR1). The cells were grown at 37° C. with CO₂ and humidity control until the desired cell density and cell number was obtained.

The medium was poured off and 10 mls/T-180 flask of enzyme-free dissociation media (Specialty Media Inc.) was added. The cells were incubated at 37° C. for 10 min or until cells sloughed off when flask was banged against hand.

The cells were harvested into 200 mL centrifuge tubes and spun at 1000 rpm, 4° C., for 10 min. The supernatant was discarded and the cells were resuspended in 5 mls/monolayer membrane preparation buffer having the composition: 10 mM Tris pH 7.2-7.4; 4 μg/mL Leupeptin (Sigma); 10 μM Phosphoramidon (Boehringer Mannheim); 40 μg/mL Bacitracin (Sigma); 5 μg/mL Aprotinin (Sigma); 10 mM Pefabloc (Boehringer Mannheim). The cells were homogenized with motor-driven dounce (Talboy setting 40), using 10 strokes and the homogenate centrifuged at 6,000 rpm, 4° C., for 15 min.

The pellets were resuspended in 0.2 mls/monolayer membrane prep buffer and aliquots were placed in tubes (500-1000 μL/tube) and quick frozen in liquid nitrogen and then stored at −80° C.

Test compounds or unlabelled NDP-α-MSH was added to 100 μL of membrane binding buffer to a final concentration of 1 μM. The membrane binding buffer had the composition: 50 mM Tris pH 7.2; 2 mM CaCl₂; 1 mM MgCl₂; 5 mM KCl; 0.2% BSA; 4 μg/mL Leupeptin (SIGMA); 10 μM Phosphoramidon (Boehringer Mannheim); 40 μg/mL Bacitracin (SIGMA); 5 μg/mL Aprotinin (SIGMA); and 10 mM Pefabloc (Boehringer Mannheim). One hundred [L of membrane binding buffer containing 10-40 μg membrane protein was added, followed by 100 μM 125]-NDP-α-MSH to final concentration of 100 μM. The resulting mixture was vortexed briefly and incubated for 90-120 min at room temp while shaking.

The mixture was filtered with Packard Microplate 196 filter apparatus using Packard Unifilter 96-well GF/C filter with 0.1% polyethyleneimine (Sigma). The filter was washed (5 times with a total of 10 mL per well) with room temperature of filter wash having the composition: 50 mM Tris-HCl pH 7.2 and 20 mM NaCl. The filter was dried, and the bottom sealed and 50 μL of Packard Microscint-20 was added to each well. The top was sealed and the radioactivity quantitated in a Packard Topcount Microplate Scintillation counter.

Melanocortin-4 receptor agonists of use in the present invention are compounds which are potent melanocortin-4 receptor agonists, i.e. compounds with an MC4R affinity (IC₅₀) of less than 300 nM, preferably less than 100 nM, and more preferably less than 45 nM.

Results of binding assay (Example 1) and selectivity for representative compounds of the present invention are provided below:

| | IC₅₀ (nM) | Binding Assay IC₅₀ (nM) | | | Selectivity Binding IC₅₀ | | |
|---|---|---|---|---|---|---|---|
| Compound | hMC4R | hMC1bR | hMC3R | hMC5R | 1R/4R | 3R/4R | 5R/4R |
| A | 44 | 5600 | 7200 | 1600 | 127.27 | 163.6 | 36.36 |
| B | 347 | 7600 | >20000 | 5250 | 21.90 | >57.64 | >15.13 |
| C | 420 | | >20000 | 3900 | | >47.62 | >9.29 |
| D | 182 | | >20000 | 4600 | | >109.89 | >25.27 |
| E | 520 | | >20000 | 6300 | | >38.46 | >12.12 |

### EXAMPLE 2

### cAMP Functional Assay

### To Discriminate Melanocortin Receptor Agonists from Antagonists

Cells (for example, CHO- or L-cells or other eukaryotic cells) expressing a human melanocortin receptor (see e.g. Yang-YK; Ollmann-MM; Wilson-BD; Dickinson-C; Yamada-T; Barsh-GS; Gantz-I; Mol-Endocrinol. 1997 March; 11(3): 274-80) were dissociated from tissue culture flasks by rinsing with Ca and Mg free phosphate buffered saline (14190-136, Life Technologies, Gaithersburg, Md.) and detached following 5 min incubation at 37° C. with enzyme free dissociation buffer (S-014-B, Specialty Media, Lavellette, N.J.). Cells were collected by centrifugation and resuspended in Earle's Balanced Salt Solution (14015-069, Life Technologies, Gaithersburg, Md.) with additions of 10 mM HEPES pH 7.5, 5 mM MgCl₂, 1 mM glutamine and 1 mg/ml bovine serum albumin. Cells were counted and diluted to 1 to 5×10⁶/mL. The phosphodiesterase inhibitor 3-isobutyl-1-methylxanthine was added to cells to 0.6 mM.

Agonist Assay: Test compounds were diluted in dimethylsulfoxide (DMSO) (10⁻⁵ to 10⁻¹⁰ M) and 0.1 volume of compound solution was added to 0.9 volumes of cell suspension; the final DMSO concentration was 1%. After room temperature incubation for 45 min, cells were lysed by incubation at 100° C. for 5 min to release accumulated cAMP.

cAMP was measured in an aliquot of the cell lysate with the Amersham (Arlington Heights, Ill.) cAMP detection assay (RPA556). The amount of cAMP production which resulted from an unknown compound was compared to that amount of cAMP produced in response to alpha-MSH which was defined as a 100% agonist. The EC₅₀ is defined as the compound concentration which results in half maximal stimulation, when compared to its own maximal level of stimulation.

Antagonist assay: Antagonist activity was defined as the ability of a compound to block cAMP production in response to alpha-MSH or other agonists. Solution of test compounds and suspension of receptor containing cells were prepared and mixed as described above; the mixture was incubated for 15 min, and an EC₅₀ dose (approximately 10 nM alpha-MSH) was added to the cells. The assay was terminated at 45 min and cAMP quantitated as above. Percent inhibition was determined by comparing the amount of cAMP produced in the presence to that produced in the absence of test compound.

Melanocortin-4 receptor agonists of use in the present invention are compounds which are potent melanocortin-4 receptor agonists, i.e. compounds with an MC4R functional activity (EC₅₀) less than 90 nM, preferably less than 40 nM, and more preferably less than 15 nM.

Results of cAMP assay (Example 2) and selectivity for representative compounds of the present invention are provided below:

| | EC50 (nM) | cAMP Assay EC50 (nM) | | | | Selectivity cAMP EC50 | | | |
|---|---|---|---|---|---|---|---|---|---|
| Compound | hMC4R | hMC1bR | hMC2R | hMC3R | hMC5R | 1R/4R | 2R/4R | 3R/4R | 5R/4R |
| A | 14 | 1700 | >10000 | 920 | 1400 | 121.43 | >714.2 | 65.71 | 100 |
| B | 88 | 1804 | | 2967 | >5000 | 20.50 | | 33.72 | >56.8 |
| C | 76 | 1100 | | 2625 | >5000 | 14.47 | | 34.54 | >65.7 |
| D | 27 | 580 | | 1500 | 3600 | 21.48 | | 55.56 | 133.3 |
| E | 120 | 1800 | | >5000 | >5000 | 15.00 | | 41.67 | 41.67 |

### EXAMPLE 3

### Maintenance Ethanol Self-Administration Following Treatment of MC4R Agonist (Compound A)

Animals: Male Wistar rats were used in all experiments. Body weight was 180-200 g at the start of the experiments. Rats were housed 2-3 per cage with food and water available ad libitum. Lights were on a 12-hour light/dark cycle, with lights on at 0600. All procedures met the guidelines of the National Institutes of Health Guide for the Care and Use of Laboratory Animals.

Operant Ethanol Self-Administration: Ethanol dilutions (5, 8 and 10% w/v) were prepared with 95% ethyl alcohol and tap water. Saccharin (Sigma Chemical Co., St. Louis, Mo.) was added to water or ethanol solutions to achieve a concentration of 0.2% w/v. Standard operant chambers (Med Associates, VT) housed in sound-attenuated chambers were used for ethanol self-administration. Syringe pumps dispensed ethanol and water into two stainless steel reservoirs mounted 4 cm above the floor of the chamber, centered between the two operant levers. The two retractable levers were located 4.5 cm to either side of the fluid reservoir. Fluid delivery and recording of operant responding were controlled by microcomputer. Rats were trained on a continuous reinforcement fixed-ratio 1 (FR1) schedule to self-administer oral alcohol (10% w/v, 0.1 ml per delivery) or water in 30 minute sessions. Rats were trained to lever press using a saccharine fading procedure whereby a lever press resulted in the delivery of a saccharin solution. Over approximately 21 days, ethanol was introduced at increasing concentrations and saccharine was removed. Once reliable levels of alcohol-reinforced self-administration were achieved, the effect of Compound (0, 5, 10 and 20 mg/kg) on maintenance levels of responding was investigated.

Results: Compound A dose dependently reduced maintenance levels of alcohol self-administration with significant reductions from baseline levels observed at 20 mg/kg (p=0.0275; t=2.093; df=14). (see FIG. 2). Water consumption was not significantly altered at any dose tested, suggesting the effect of Compound A on response rates was specific to alcohol and not due to a general reduction in operant response rates.

### EXAMPLE 4

### Attenuation of Alcohol Deprivation (ADE) Effect Following Infusion of MC4R Agonist (Compound A)

Method: Methods for this experiment were as described in EXAMPLE 3. Following the maintenance ethanol self-administration experiment, rats were re-baselined and once reliable levels of alcohol-reinforced self-administration were achieved, rats were confined to their home cages for a 10-day period. Prior to reintroduction to the test chamber, animals were injected with Compound (0, 5, 10 and 20 mg/kg).

Results: Analysis of Variance revealed an overall significant effect of dose (F [4.47]=7.6, p=0.0001). A dose dependent effect was observed in the ADE experiment with 5 and 10 mg/kg blocking the ADE and 20 mg/kg leading to a significant decrease below baseline levels of consumption (see FIG. 3). Water consumption was not significantly altered at any dose tested, suggesting the effect of Compound A on response rates was specific to alcohol and not due to a general reduction in operant response rates.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the subject being treated for any of the indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1-19. (canceled)

20. A method selected from: a method of reducing alcohol consumption, a method of inhibiting alcohol consumption, a method of inhibiting the alcohol deprivation effect, a method of treating alcoholism and a method of treating alcohol abuse, comprising administering a therapeutically effective amount of a non-peptidyl melanocortin 4 receptor agonist to a subject in need thereof.

21. The method of reducing alcohol consumption according to claim 20 comprising administering a therapeutically effective amount of a non-peptidyl melanocortin 4 receptor agonist to a subject in need thereof.

22. The method of claim 21 wherein the non-peptidyl melanocortin 4 receptor agonist is a selective melanocortin 4 receptor agonist.

23. The method of claim 21 wherein the melanocortin 4 receptor agonist is an orally active melanocortin 4 receptor agonist.

24. The method of claim 22 wherein the melanocortin 4 receptor agonist has a selective functional activity characterized by an EC₅₀ at least 65-fold lower for the human melanocortin 4 receptor than for the human melanocortin 1 receptor, the melanocortin 2 receptor, the human melanocortin 3 receptor and the human melanocortin 5 receptor.

25. The method of claim 24 wherein the functional activity of the selective melanocortin 4 receptor agonist is characterized by an EC₅₀ at least 120-fold lower for the human melanocortin 4 receptor than for the human melanocortin 1 receptor.

26. The method of claim 24 wherein the functional activity of the selective melanocortin 4 receptor agonist is characterized by an EC₅₀ at least 700-fold lower for the human melanocortin 4 receptor than for the human melanocortin 2 receptor.

27. The method of claim 24 wherein the functional activity of the selective melanocortin 4 receptor agonist is characterized by an EC₅₀ at least 90-fold lower for the human melanocortin 4 receptor than for the human melanocortin 5 receptor.

28. The method of claim 24 wherein the selective melanocortin 4 receptor agonist has a binding affinity index IC₅₀ value of less than 45 nM at the human melanocortin 4 receptor.

29. The method of claim 21 wherein the melanocortin 4 receptor agonist is a compound of Formula I or II: 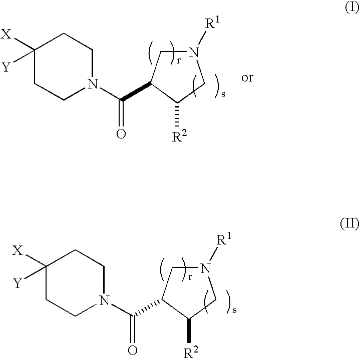
or a pharmaceutically acceptable salt thereof; wherein
X is selected from the group consisting of: C₁₋₈ alkyl, (CH₂)ₙC₃₋₈ cycloalkyl, (CH₂)ₙ-phenyl, (CH₂)ₙ-naphthyl, (CH₂)ₙ-heteroaryl, (CH₂)ₙheterocyclyl, (CH₂)ₙC≡N, (CH₂)ₙCON(R⁵R⁵), (CH₂)ₙCO₂R⁵, (CH₂)ₙCOR⁵, (CH₂)ₙNR⁵C(O)R⁵, (CH₂)ₙNR⁵CO₂R⁵, (CH₂)ₙNR⁵C(O)N(R⁵)₂, (CH₂)ₙNR⁵SO₂R⁵, (CH₂)ₙS(O)ₚR⁵, (CH₂)ₙSO₂N(R⁵)(R⁵), (CH₂)ₙOR⁵, (CH₂)ₙOC(O)R⁵, (CH₂)ₙOC(O)OR⁵, (CH₂)ₙOC(O)N(R⁵)₂, (CH₂)ₙN(R⁵)(R⁵), and (CH₂)ₙNR⁵SO₂N(R⁵)(R⁵), wherein heteroaryl is as defined above; phenyl, naphthyl, and heteroaryl are unsubstituted or substituted with one to three groups independently selected from R⁶; alkyl, cycloalkyl, and heterocyclyl are unsubstituted or substituted with one to three groups independently selected from R⁶ and oxo; and wherein any methylene (CH₂) in X is unsubstituted or substituted with one to two groups independently selected from halogen, hydroxy, and C₁₋₄ alkyl;
Y is selected from the group consisting of: hydrogen, C₁₋₈ alkyl, C₂₋₆ alkenyl, (CH₂)ₙC₃₋₈ cycloalkyl, (CH₂)ₙ-phenyl, (CH₂)ₙ-naphthyl, (CH₂)ₙ-heteroaryl, and (CH₂)ₙ-heterocyclyl, wherein heteroaryl is as defined above, and phenyl, naphthyl, and heteroaryl are unsubstituted or substituted with one to three groups independently selected from R⁶; alkyl, cycloalkyl, and heterocyclyl are optionally substituted with one to three groups independently selected from
R⁶ and oxo; and wherein any methylene (CH₂) in Y is unsubstituted or substituted with one to two groups independently selected from halogen, hydroxy, and C₁₋₄ alkyl;
R¹ is selected from the group consisting of: hydrogen, amidino, C₁₋₄ alkyliminoyl, C₁₋₁₀ alkyl, (CH₂)ₙ—C₃₋₇ cycloalkyl, (CH₂)ₙ-phenyl, (CH₂)ₙ-naphthyl, and (CH₂)ₙ-heteroaryl, wherein heteroaryl is selected from the group consisting of (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³; and alkyl and cycloalkyl are unsubstituted or substituted with one to three groups independently selected from R³ and oxo;
R² is selected from the group consisting of: phenyl, naphthyl, and heteroaryl, wherein heteroaryl is selected from the group consisting of: (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³;
each R³ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃, in which heteroaryl is as defined above; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
each R⁴ is independently selected from the group consisting of: hydrogen, C₁₋₆ alkyl, (CH₂)ₙ phenyl, (CH₂)ₙ-heteroaryl, (CH₂)ₙ-naphthyl, (CH₂)ₙ-heterocyclyl, (CH₂)ₙC₃₋₇ cycloalkyl, and (CH₂)ₙC₃₋₇ bicycloalkyl, wherein alkyl, phenyl, heteroaryl, heterocyclyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl;
each R⁵ is independently selected from the group consisting of: hydrogen, C₁₋₈ alkyl, (CH₂)ₙ-phenyl, (CH₂)ₙ-naphthyl, (CH₂)ₙ-heteroaryl, and (CH₂)ₙC₃₋₇ cycloalkyl, wherein heteroaryl is as defined above; phenyl, naphthyl, and heteroaryl are unsubstituted or substituted with one to three groups independently selected from R³; alkyl and cycloalkyl are unsubstituted or substituted with one to three groups independently selected from R³ and oxo; and wherein any methylene (CH₂) in R⁵ is unsubstituted or substituted with one to two groups independently selected from halogen, hydroxy, and C₁₋₄ alkyl; or two R⁵ groups together with the atom to which they are attached form a 5- to 8-membered mono- or bicyclic ring system optionally containing an additional heteroatom selected from O, S, and NC₁₋₄ alkyl;
each R⁶ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃, in which heteroaryl is as defined above; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R⁶ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
r is 1 or 2;
s is 0, 1, or 2;
n is 0, 1 or 2; and
p is 0, 1, or 2.

30. The method of claim 29 wherein X is phenyl substituted with two groups independently selected from R³, Y is hydrogen, and R¹ is tert-butyl, and R² is phenyl substituted with two groups independently selected from R³, or a pharmaceutically acceptable salt thereof.

31. The method of claim 30 wherein the melanocortin 4 receptor agonist of Formula I is 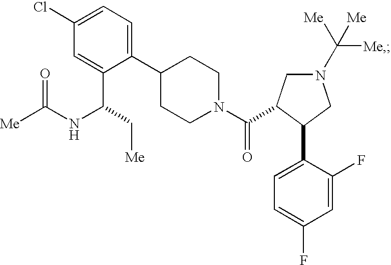
or a pharmaceutically acceptable salt thereof.

32. The method of inhibiting alcohol consumption according to claim 20 comprising administering a non-peptidyl melanocortin 4 receptor agonist, or a pharmaceutically acceptable salt thereof, to a subject in need thereof wherein the melanocortin 4 receptor agonist is a compound of Formula I or II: 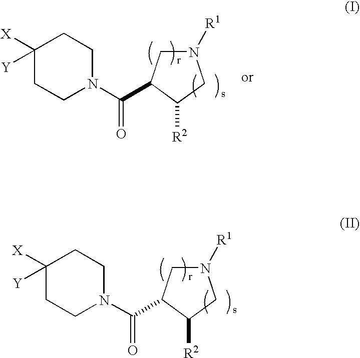
or a pharmaceutically acceptable salt thereof; wherein
X is selected from the group consisting of: C₁₋₈ alkyl, (CH₂)ₙC₃₋₈ cycloalkyl, (CH₂)ₙ-phenyl, (CH₂)ₙ-naphthyl, (CH₂)ₙ-heteroaryl, (CH₂)ₙheterocyclyl, (CH₂)ₙC≡N, (CH₂)ₙCON(R⁵R⁵), (CH₂)ₙCO₂R⁵, (CH₂)ₙCOR⁵, (CH₂)ₙNR⁵C(O)R⁵, (CH₂)ₙNR⁵CO₂R⁵, (CH₂)ₙNR⁵C(O)N(R⁵)₂, (CH₂)ₙNR⁵SO₂R⁵, (CH₂)ₙS(O)ₚR⁵, (CH₂)ₙSO₂N(R⁵)(R⁵), (CH₂)ₙOR⁵, (CH₂)ₙOC(O)R⁵, (CH₂)ₙOC(O)OR⁵, (CH₂)ₙOC(O)N(R⁵)₂, (CH₂)ₙN(R⁵)(R⁵), and (CH₂)ₙNR⁵SO₂N(R⁵)(R⁵), wherein heteroaryl is as defined above; phenyl, naphthyl, and heteroaryl are unsubstituted or substituted with one to three groups independently selected from R⁶; alkyl, cycloalkyl, and heterocyclyl are unsubstituted or substituted with one to three groups independently selected from R⁶ and oxo; and wherein any methylene (CH₂) in X is unsubstituted or substituted with one to two groups independently selected from halogen, hydroxy, and C₁₋₄ alkyl;
Y is selected from the group consisting of: hydrogen, C₁₋₈ alkyl, C₂₋₆ alkenyl, (CH₂)ₙC₃₋₈ cycloalkyl, (CH₂)ₙ-phenyl, (CH₂)ₙ-naphthyl, (CH₂)ₙ-heteroaryl, and (CH₂)ₙ-heterocyclyl, wherein heteroaryl is as defined above, and phenyl, naphthyl, and heteroaryl are unsubstituted or substituted with one to three groups independently selected from R⁶; alkyl, cycloalkyl, and heterocyclyl are optionally substituted with one to three groups independently selected from R⁶ and oxo; and wherein any methylene (CH₂) in Y is unsubstituted or substituted with one to two groups independently selected from halogen, hydroxy, and C₁₋₄ alkyl;
R¹ is selected from the group consisting of: hydrogen, amidino, C₁₋₄ alkyliminoyl, C₁₋₁₀ alkyl, (CH₂)ₙ—C₃₋₇ cycloalkyl, (CH₂)ₙ-phenyl, (CH₂)ₙ-naphthyl, and (CH₂)ₙ-heteroaryl, wherein heteroaryl is selected from the group consisting of (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³; and alkyl and cycloalkyl are unsubstituted or substituted with one to three groups independently selected from R³ and oxo;
R² is selected from the group consisting of: phenyl, naphthyl, and heteroaryl, wherein heteroaryl is selected from the group consisting of: (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³;
each R³ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃, in which heteroaryl is as defined above; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
each R⁴ is independently selected from the group consisting of: hydrogen, C₁₋₆ alkyl, (CH₂)ₙ phenyl, (CH₂)ₙ-heteroaryl, (CH₂)ₙ-naphthyl, (CH₂)ₙ-heterocyclyl, (CH₂)ₙC₃₋₇ cycloalkyl, and (CH₂)ₙC₃₋₇ bicycloalkyl, wherein alkyl, phenyl, heteroaryl, heterocyclyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl;
each R⁵ is independently selected from the group consisting of: hydrogen, C₁₋₈ alkyl, (CH₂)ₙ-phenyl, (CH₂)ₙ-naphthyl, (CH₂)ₙ-heteroaryl, and (CH₂)ₙC₃₋₇ cycloalkyl, wherein heteroaryl is as defined above; phenyl, naphthyl, and heteroaryl are unsubstituted or substituted with one to three groups independently selected from R³; alkyl and cycloalkyl are unsubstituted or substituted with one to three groups independently selected from R³ and oxo; and wherein any methylene (CH₂) in R⁵ is unsubstituted or substituted with one to two groups independently selected from halogen, hydroxy, and C₁₋₄ alkyl; or two R⁵ groups together with the atom to which they are attached form a 5- to 8-membered mono- or bicyclic ring system optionally containing an additional heteroatom selected from O, S, and NC₁₋₄ alkyl;
each R⁶ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃, in which heteroaryl is as defined above; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R⁶ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
r is 1 or 2;
s is 0, 1, or 2;
n is 0, 1 or 2; and
p is 0, 1, or 2.

33. The method of inhibiting the alcohol deprivation effect in a subject in need thereof according to claim 20 comprising administering a non-peptidyl melanocortin 4 receptor agonist, or a pharmaceutically acceptable salt thereof, to a subject in need thereof wherein the melanocortin 4 receptor agonist is a compound of Formula I or II: 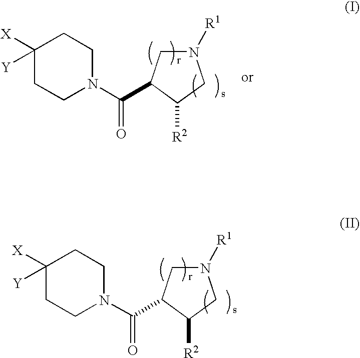
or a pharmaceutically acceptable salt thereof; wherein
X is selected from the group consisting of: C₁₋₈ alkyl, (CH₂)ₙC₃₋₈ cycloalkyl, (CH₂)ₙ-phenyl, (CH₂)ₙ-naphthyl, (CH₂)ₙ-heteroaryl, (CH₂)ₙheterocyclyl, (CH₂)ₙC≡N, (CH₂)ₙCON(R⁵R⁵), (CH₂)ₙCO₂R⁵, (CH₂)ₙCOR⁵, (CH₂)ₙNR⁵C(O)R⁵, (CH₂)ₙNR⁵CO₂R⁵, (CH₂)ₙNR⁵C(O)N(R⁵)₂, (CH₂)ₙNR⁵SO₂R⁵, (CH₂)ₙS(O)ₚR⁵, (CH₂)ₙSO₂N(R⁵)(R⁵), (CH₂)ₙOR⁵, (CH₂)ₙOC(O)R⁵, (CH₂)ₙOC(O)OR⁵, (CH₂)ₙOC(O)N(R⁵)₂, (CH₂)ₙN(R⁵)(R⁵), and (CH₂)ₙNR⁵SO₂N(R⁵)(R⁵), wherein heteroaryl is as defined above; phenyl, naphthyl, and heteroaryl are unsubstituted or substituted with one to three groups independently selected from R⁶; alkyl, cycloalkyl, and heterocyclyl are unsubstituted or substituted with one to three groups independently selected from R⁶ and oxo; and wherein any methylene (CH₂) in X is unsubstituted or substituted with one to two groups independently selected from halogen, hydroxy, and C₁₋₄ alkyl;
Y is selected from the group consisting of: hydrogen, C₁₋₈ alkyl, C₂₋₆ alkenyl, (CH₂)ₙC₃₋₈ cycloalkyl, (CH₂)ₙ-phenyl, (CH₂)ₙ-naphthyl, (CH₂)ₙ-heteroaryl, and (CH₂)ₙ-heterocyclyl, wherein heteroaryl is as defined above, and phenyl, naphthyl, and heteroaryl are unsubstituted or substituted with one to three groups independently selected from R⁶; alkyl, cycloalkyl, and heterocyclyl are optionally substituted with one to three groups independently selected from R⁶ and oxo; and wherein any methylene (CH₂) in Y is unsubstituted or substituted with one to two groups independently selected from halogen, hydroxy, and C₁₋₄ alkyl;
R¹ is selected from the group consisting of: hydrogen, amidino, C₁₋₄ alkyliminoyl, C₁₋₁₀ alkyl, (CH₂)ₙ—C₃₋₇ cycloalkyl, (CH₂)ₙ-phenyl, (CH₂)ₙ-naphthyl, and (CH₂)ₙ-heteroaryl, wherein heteroaryl is selected from the group consisting of (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³; and alkyl and cycloalkyl are unsubstituted or substituted with one to three groups independently selected from R³ and oxo;
R² is selected from the group consisting of: phenyl, naphthyl, and heteroaryl, wherein heteroaryl is selected from the group consisting of: (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³;
each R³ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃, in which heteroaryl is as defined above; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
each R⁴ is independently selected from the group consisting of: hydrogen, C₁₋₆ alkyl, (CH₂)ₙ phenyl, (CH₂)ₙ-heteroaryl, (CH₂)ₙ-naphthyl, (CH₂)ₙ-heterocyclyl, (CH₂)ₙC₃₋₇ cycloalkyl, and (CH₂)ₙC₃₋₇ bicycloalkyl, wherein alkyl, phenyl, heteroaryl, heterocyclyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl;
each R⁵ is independently selected from the group consisting of: hydrogen, C₁₋₈ alkyl, (CH₂)ₙ-phenyl, (CH₂)ₙ-naphthyl, (CH₂)ₙ-heteroaryl, and (CH₂)ₙC₃₋₇ cycloalkyl, wherein heteroaryl is as defined above; phenyl, naphthyl, and heteroaryl are unsubstituted or substituted with one to three groups independently selected from R³; alkyl and cycloalkyl are unsubstituted or substituted with one to three groups independently selected from R³ and oxo; and wherein any methylene (CH₂) in R⁵ is unsubstituted or substituted with one to two groups independently selected from halogen, hydroxy, and C₁₋₄ alkyl; or two R⁵ groups together with the atom to which they are attached form a 5- to 8-membered mono- or bicyclic ring system optionally containing an additional heteroatom selected from O, S, and NC₁₋₄ alkyl;
each R⁶ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃, in which heteroaryl is as defined above; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R⁶ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
r is 1 or 2;
s is 0, 1, or 2;
n is 0, 1 or 2; and
p is 0, 1, or 2.

34. The method of treating alcoholism according to claim 20 comprising administering a non-peptidyl melanocortin 4 receptor agonist, or a pharmaceutically acceptable salt thereof, to a subject in need thereof wherein the melanocortin 4 receptor agonist is a compound of Formula I or II: 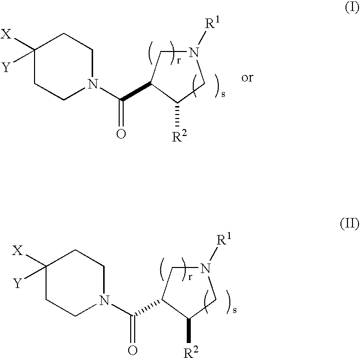
or a pharmaceutically acceptable salt thereof; wherein
X is selected from the group consisting of: C₁₋₈ alkyl, (CH₂)ₙC₃₋₈ cycloalkyl, (CH₂)ₙ-phenyl, (CH₂)ₙ-naphthyl, (CH₂)ₙ-heteroaryl, (CH₂)ₙheterocyclyl, (CH₂)ₙC≡N, (CH₂)ₙCON(R⁵R⁵), (CH₂)ₙCO₂R⁵, (CH₂)ₙCOR⁵, (CH₂)ₙNR⁵C(O)R⁵, (CH₂)ₙNR⁵CO₂R⁵, (CH₂)ₙNR⁵C(O)N(R⁵)₂, (CH₂)ₙNR⁵SO₂R⁵, (CH₂)ₙS(O)ₚR⁵, (CH₂)ₙSO₂N(R⁵)(R⁵), (CH₂)ₙOR⁵, (CH₂)ₙOC(O)R⁵, (CH₂)ₙOC(O)OR⁵, (CH₂)ₙOC(O)N(R⁵)₂, (CH₂)ₙN(R⁵)(R⁵), and (CH₂)ₙNR⁵SO₂N(R⁵)(R⁵), wherein heteroaryl is as defined above; phenyl, naphthyl, and heteroaryl are unsubstituted or substituted with one to three groups independently selected from R⁶; alkyl, cycloalkyl, and heterocyclyl are unsubstituted or substituted with one to three groups independently selected from R⁶ and oxo; and wherein any methylene (CH₂) in X is unsubstituted or substituted with one to two groups independently selected from halogen, hydroxy, and C₁₋₄ alkyl;
Y is selected from the group consisting of: hydrogen, C₁₋₈ alkyl, C₂₋₆ alkenyl, (CH₂)ₙC₃₋₈ cycloalkyl, (CH₂)ₙ-phenyl, (CH₂)ₙ-naphthyl, (CH₂)ₙ-heteroaryl, and (CH₂)ₙ-heterocyclyl, wherein heteroaryl is as defined above, and phenyl, naphthyl, and heteroaryl are unsubstituted or substituted with one to three groups independently selected from R⁶; alkyl, cycloalkyl, and heterocyclyl are optionally substituted with one to three groups independently selected from R⁶ and oxo; and wherein any methylene (CH₂) in Y is unsubstituted or substituted with one to two groups independently selected from halogen, hydroxy, and C₁₋₄ alkyl;
R¹ is selected from the group consisting of: hydrogen, amidino, C₁₋₄ alkyliminoyl, C₁₋₁₀ alkyl, (CH₂)ₙ—C₃₋₇ cycloalkyl, (CH₂)ₙ-phenyl, (CH₂)ₙ-naphthyl, and (CH₂)ₙ-heteroaryl, wherein heteroaryl is selected from the group consisting of (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³; and alkyl and cycloalkyl are unsubstituted or substituted with one to three groups independently selected from R³ and oxo;
R² is selected from the group consisting of: phenyl, naphthyl, and heteroaryl, wherein heteroaryl is selected from the group consisting of: (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³;
each R³ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃, in which heteroaryl is as defined above; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
each R⁴ is independently selected from the group consisting of: hydrogen, C₁₋₆ alkyl, (CH₂)ₙ phenyl, (CH₂)ₙ-heteroaryl, (CH₂)ₙ-naphthyl, (CH₂)ₙ-heterocyclyl, (CH₂)ₙC₃₋₇ cycloalkyl, and (CH₂)ₙC₃₋₇ bicycloalkyl, wherein alkyl, phenyl, heteroaryl, heterocyclyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl;
each R⁵ is independently selected from the group consisting of: hydrogen, C₁₋₈ alkyl, (CH₂)ₙ-phenyl, (CH₂)ₙ-naphthyl, (CH₂)ₙ-heteroaryl, and (CH₂)ₙC₃₋₇ cycloalkyl, wherein heteroaryl is as defined above; phenyl, naphthyl, and heteroaryl are unsubstituted or substituted with one to three groups independently selected from R³; alkyl and cycloalkyl are unsubstituted or substituted with one to three groups independently selected from R³ and oxo; and wherein any methylene (CH₂) in R⁵ is unsubstituted or substituted with one to two groups independently selected from halogen, hydroxy, and C₁₋₄ alkyl; or two R⁵ groups together with the atom to which they are attached form a 5- to 8-membered mono- or bicyclic ring system optionally containing an additional heteroatom selected from O, S, and NC₁₋₄ alkyl;
each R⁶ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃, in which heteroaryl is as defined above; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R⁶ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
r is 1 or 2;
s is 0, 1, or 2;
n is 0, 1 or 2; and
p is 0, 1, or 2.

35. The method of treating alcohol abuse according to claim 20 comprising administering a non-peptidyl melanocortin 4 receptor agonist, or a pharmaceutically acceptable salt thereof, to a subject in need thereof wherein the melanocortin 4 receptor agonist is a compound of Formula I or II: 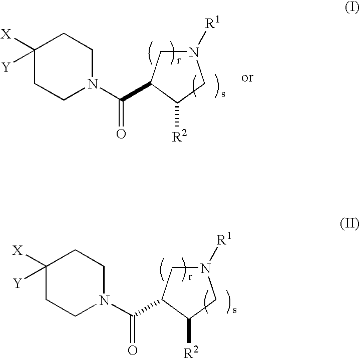
or a pharmaceutically acceptable salt thereof; wherein
X is selected from the group consisting of: C₁₋₈ alkyl, (CH₂)ₙC₃₋₈ cycloalkyl, (CH₂)ₙ-phenyl, (CH₂)ₙ-naphthyl, (CH₂)ₙ-heteroaryl, (CH₂)ₙheterocyclyl, (CH₂)ₙC≡N, (CH₂)ₙCON(R⁵R⁵), (CH₂)ₙCO₂R⁵, (CH₂)ₙCOR⁵, (CH₂)ₙNR⁵C(O)R⁵, (CH₂)ₙNR⁵CO₂R⁵, (CH₂)ₙNR⁵C(O)N(R⁵)₂, (CH₂)ₙNR⁵SO₂R⁵, (CH₂)ₙS(O)ₚR⁵, (CH₂)ₙSO₂N(R⁵)(R⁵), (CH₂)ₙOR⁵, (CH₂)ₙOC(O)R⁵, (CH₂)ₙOC(O)OR⁵, (CH₂)ₙOC(O)N(R⁵)₂, (CH₂)ₙN(R⁵)(R⁵), and (CH₂)ₙNR⁵SO₂N(R⁵)(R⁵), wherein heteroaryl is as defined above; phenyl, naphthyl, and heteroaryl are unsubstituted or substituted with one to three groups independently selected from R⁶; alkyl, cycloalkyl, and heterocyclyl are unsubstituted or substituted with one to three groups independently selected from R⁶ and oxo; and wherein any methylene (CH₂) in X is unsubstituted or substituted with one to two groups independently selected from halogen, hydroxy, and C₁₋₄ alkyl;
Y is selected from the group consisting of: hydrogen, C₁₋₈ alkyl, C₂₋₆ alkenyl, (CH₂)ₙC₃₋₈ cycloalkyl, (CH₂)ₙ-phenyl, (CH₂)ₙ-naphthyl, (CH₂)ₙ-heteroaryl, and (CH₂)ₙ-heterocyclyl, wherein heteroaryl is as defined above, and phenyl, naphthyl, and heteroaryl are unsubstituted or substituted with one to three groups independently selected from R⁶; alkyl, cycloalkyl, and heterocyclyl are optionally substituted with one to three groups independently selected from R⁶ and oxo; and wherein any methylene (CH₂) in Y is unsubstituted or substituted with one to two groups independently selected from halogen, hydroxy, and C₁₋₄ alkyl;
R¹ is selected from the group consisting of: hydrogen, amidino, C₁₋₄ alkyliminoyl, C₁₋₁₀ alkyl, (CH₂)ₙ—C₃₋₇ cycloalkyl, (CH₂)ₙ-phenyl, (CH₂)ₙ-naphthyl, and (CH₂)ₙ-heteroaryl, wherein heteroaryl is selected from the group consisting of (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³; and alkyl and cycloalkyl are unsubstituted or substituted with one to three groups independently selected from R³ and oxo;
R² is selected from the group consisting of: phenyl, naphthyl, and heteroaryl, wherein heteroaryl is selected from the group consisting of: (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³;
each R³ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃, in which heteroaryl is as defined above; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
each R⁴ is independently selected from the group consisting of: hydrogen, C₁₋₆ alkyl, (CH₂)ₙ phenyl, (CH₂)ₙ-heteroaryl, (CH₂)ₙ-naphthyl, (CH₂)ₙ-heterocyclyl, (CH₂)ₙC₃₋₇ cycloalkyl, and (CH₂)ₙC₃₋₇ bicycloalkyl, wherein alkyl, phenyl, heteroaryl, heterocyclyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl;
each R⁵ is independently selected from the group consisting of: hydrogen, C₁₋₈ alkyl, (CH₂)ₙ-phenyl, (CH₂)ₙ-naphthyl, (CH₂)ₙ-heteroaryl, and (CH₂)ₙC₃₋₇ cycloalkyl, wherein heteroaryl is as defined above; phenyl, naphthyl, and heteroaryl are unsubstituted or substituted with one to three groups independently selected from R³; alkyl and cycloalkyl are unsubstituted or substituted with one to three groups independently selected from R³ and oxo; and wherein any methylene (CH₂) in R⁵ is unsubstituted or substituted with one to two groups independently selected from halogen, hydroxy, and C₁₋₄ alkyl; or two R⁵ groups together with the atom to which they are attached form a 5- to 8-membered mono- or bicyclic ring system optionally containing an additional heteroatom selected from O, S, and NC₁₋₄ alkyl;
each R⁶ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen,
OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃, in which heteroaryl is as defined above; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R⁶ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
r is 1 or 2;
s is 0, 1, or 2;
n is 0, 1 or 2; and
p is 0, 1, or 2.
